(19) United States
(12) Patent Application Publication
(10) Pub. No.: US 2024/0167999 A1
(43) Pub. Date: May 23, 2024

(54) FIELD MONITORING AND HUSBANDRY SYSTEMS AND METHODS FOR SAME

(71) Applicant: Jared Ernet Kocer, Sioux Falls, SD (US)

(72) Inventor: Jared Ernet Kocer, Sioux Falls, SD (US)

(21) Appl. No.: 18/513,333

(22) Filed: Nov. 17, 2023

Related U.S. Application Data
(60) Provisional application No. 63/384,280, filed on Nov. 18, 2022.

(51) Int. Cl. G01N 33/24

(52) U.S. Cl. CPC G01N 33/24; A01M 7/0089; G01N 2033/245

(57) ABSTRACT

A field monitoring and husbandry system is configured for mounting with one or more of an agricultural vehicle or an agricultural implement. The system includes at least one field sensor. The at least one field sensor is configured to measure one or more field characteristics within an agricultural field. The system includes a field husbandry controller in communication with the at least one field sensor. In an example, the field husbandry controller includes a map generation module configured to generate a map of at least the measurements of the at least one field sensor indexed with the one or more associated portions of the field. In another example, the field husbandry controller includes a field characteristic comparator configured to compare the one or more field characteristics with an associated field threshold of one or more field thresholds. The system optionally applies agricultural product(s) based on the comparison.

### CLAIM OF PRIORITY

This patent application claims the benefit of priority of Kocer, U.S. Provisional Patent Application Ser. No. 63/384,280, entitled “FIELD MONITORING AND HUSBANDRY SYSTEM AND METHODS FOR SAME,” filed on Nov. 18, 2022 (Attorney Docket No. 2754.503PRV), which is hereby incorporated by reference herein in its entirety.

### TECHNICAL FIELD

This document pertains generally, but not by way of limitation, to field monitoring and husbandry.

### BACKGROUND

In an example, an agricultural product (e.g., fertilizer, agricultural slurry, water, or the like) is applied to soil in an agricultural field. In another example, the agricultural product is applied to the soil at the beginning of a growing season. The agricultural product changes characteristics of the soil, for instance to enhance growth of crops in the soil. In yet another example, the characteristics of the soil are measured before applying the agricultural product, for instance with field installed sensors, soil samples taken from the field or the like. For instance, the characteristics of the soil are measured during a season, and the agricultural product is applied to the soil during the season (e.g., same or later season) to change the characteristics of the soil based on the measured characteristics of the soil.

### SUMMARY

The present inventors have recognized, among other things, that a problem to be solved includes measuring field characteristics (e.g., one or more of nitrogen content, phosphorous content, potassium content, ammonia content, ammonium content, moisture content, color of foliage, salinity, pH level, temperature or the like) of field soil (or crops growing in the field soil). In some approaches, an individual will obtain samples of the soil in one or more different locations within the agricultural field, for instance with a shovel. Accordingly, resolution of the characteristics of the soil are limited by the number of samples obtained, size of the field, variations in terrain, effort applied to collect samples or the like.

The present subject matter provides a solution to this problem, for example with a field monitoring and husbandry system. For example, the field monitoring and husbandry system includes at least one field sensor, and the at least one field sensor measures the field characteristics. In another example, the field sensor is coupled with a vehicle or implement (and may include a plurality of field sensors), and the field sensor measures the field characteristics as the vehicle moves through the field. Accordingly, the field monitoring and husbandry system including the vehicle or implement coupled field sensor (or sensors) that is navigated through the field measures field characteristics with enhanced resolution in comparison to individually obtained soil samples, static soil sensors or the like).

In another example, the field monitoring and husbandry system applies one or more agricultural products to the field (e.g., soil, crops, weeds, pests or the like). For instance, the field monitoring and husbandry system includes an agricultural product applicator, and the system controls the agricultural product applicator to apply the agricultural product to the field, such as the soil, crops, weeds, pests or the like based on sensing and analysis of field characteristics. The field monitoring and husbandry system uses the measured field characteristics (measured with the one or more field sensors) to provide a prescription for applying the one or more agricultural products to achieve a specified value (or values) (e.g., field threshold values, target values, or the like) of the field characteristics, for instance of the soil. For example, the prescription is determined or indexed relative to an actual location where the field characteristics were measured (as opposed to a generic prescription applied to the field based on soil samples obtained by an individual or statically located sensors conducting static sensing). Accordingly, the prescription varies based on the measured field characteristics (and location of the measured field characteristics in the field). The field monitoring and husbandry system changes (e.g., conditions, controls, treats, maintains, modulates, amends, or the like) the field characteristics by applying the one or more agricultural products to achieve the prescribed value(s) for the field characteristics. Thus, the field monitoring and husbandry system enhances correspondence (e.g., decreases deviation) between the measured field characteristics and one or more specified values of field characteristics by applying the prescribed agricultural products to the field based on the prescriptions and thereby changing the field characteristics to the specified values.

In still yet another example, the field monitoring and husbandry system applies the agricultural product based on the measured field characteristics. For example, the system applies the agricultural product at an application rate (e.g., according to the prescription) based on the measured field characteristics. In some examples, the system compares the measured field characteristics with a field threshold, such as a specified value of one or more field characteristics. The system generates the application rate for the agricultural product based on the comparison of the measured field characteristics with the field threshold. Accordingly, the system applies the agricultural product to the field to change (control) and guide the field characteristics toward the specified field threshold, also referred to as one or more specified values of field characteristics. Thus, the system applies agricultural product to decrease a difference (including, minimizing, guiding to a deviation of zero, decreasing a difference or the like) between field characteristics and the field threshold.

The present inventors have recognized, among other things, that a problem to be solved includes monitoring of yield values for crops grown in the agricultural field and using yield values to enhance husbandry. For example, yield values for the crops grown in the field vary within the field (e.g., by zones) and vary with each growing season. In some approaches, systems monitoring yield values refrain from monitoring (or logging) agricultural product applications to the field before, during or after a growing season relative to yield values.

The present subject matter provides a solution to this problem, for example with the field monitoring and husbandry system. In this example, the field monitoring and husbandry system includes a yield monitor. The yield monitor measures one or more yield values for crops grown in the agricultural field. In another example, the field monitoring and husbandry system indexes the yield values with one or more of measured field characteristics or controlled field characteristics. For instance, the system indexes a yield value for a portion or zone of the field with (measured or controlled) field characteristics for the portion or zone of the field having that yield value. Accordingly, the system monitors (or logs) the field characteristics for a portion of the field and associates the monitored field characteristics with the yield value from that portion of the field. Thus, the field monitoring and husbandry system monitors yield values for portions of the field and field characteristics that assisted with achieving corresponding yield values.

In yet another example, the field monitoring and husbandry system uses the yield value as an input for modulation of the field characteristics. For instance, the system updates field thresholds for portions or zones (herein portions) of the field based on one or more measured yield values. In an example, the field monitoring and husbandry system determines a favored yield value based on one or more of the yield values (measured with the yield monitor) or the field characteristics (measured with the field sensors). For example, the system selects a favored yield value (from a portion of the field). In another example, the system collects an array of the greatest yield values from the field and compares those yield values and the agricultural products applied in those same zones. The system selects the greatest yield of the array having the least costly set of associated agricultural products applied as the favored yield value. The system provides field characteristics for the portion of the field 106 associated with the favored yield value. Accordingly, the system is capable of indexing the field characteristics associated with the favored yield value. As noted below the tracked relationship between the favored yield value and the associated field characteristics facilitates enhanced husbandry with a goal of increasing yield.

The system optionally imputes the field characteristics associated with the favored yield value to other portions of the field by updating one or more field thresholds to correspond with the field characteristics associated with the favored yield value. In one example, the system optionally updates or revises at least one field threshold for the ‘other’ portions of the field to correspond with the field characteristics for the portion of the agricultural field having the favored yield value. Accordingly, the system uses the favored yield value and its associated field characteristics to refine or update field characteristics (e.g., product application or the like) and enhance yield in other portions of the field, such as previously underperforming portions. Thus, other portions of the field that had different yields than the favored yield value have their prescriptions (e.g., field threshold values and agricultural product applications) updated to guide the field characteristics for those other portions toward the field characteristics associated with the favored yield value. Consequently, the system applies agricultural product, conducts cultivating or the like based on (favored) yield values for the agricultural field and imputation of associated field characteristics to other portions of the field to enhance crop growth and corresponding yield across the field.

The present inventors have recognized, among other things, that a problem to be solved includes enhancing one or both of accuracy or precision of field characteristics measured with a field sensor. For instance, the at least one field sensor includes an above-ground field sensor that measures field characteristics. This type of field sensor uses above-ground sensors to measure field characteristics, for instance of features above the surface (e.g., foliage of crops, or the like), at the surface, or below the surface of the soil (e.g., such as nutrients, water or the like). In some approaches, the above-ground field sensor has limited accuracy, precision or both relative to a below-ground field sensor. Below-ground field sensors measure field characteristics at or below the surface of the soil. For instance, the below-ground field sensor directly contacts soil and provides enhanced accuracy or precision of field characteristic measurements relative to above-ground sensors. However, in some examples, the below-ground field sensor is driven into a location in the field, is accordingly stationary in the field, and thereby does not provide measurements throughout the field.

Conversely, the above-ground field sensor is optionally mounted on a vehicle (or an agricultural implement) that traverses the field. The above-ground field sensor measures above-ground field characteristics as the vehicle traverses the field. As noted herein the field characteristic measurements are readily indexed to the associated portions of the field. Accordingly, the above-ground field sensor facilitates high resolution enhanced indexing of measurements of field characteristics (e.g., resolution) to portions of the field in comparison to static sensors.

The present subject matter provides a solution to this problem, for example with the field monitoring and husbandry system including above-ground field sensors that are calibrated based on below-ground sensing while at the same time are navigated through the field to provide high resolution measurements. In an example, the field monitoring and husbandry system calibrates a field sensor, for instance the above-ground field sensor with below-ground measurements. For example, the system marries measurements of the below-ground field sensor with corresponding measurements of the above-ground field sensor to provide a measurement compensation value that adjusts the above-ground measurements in a manner commensurate to the (more precise or accurate) below-ground sensing. The system uses measured below-ground field characteristics to calibrate measured above-ground field characteristics. For example, the system compares the above-ground field characteristics with the below-ground field characteristics to determine a sensor deviation. The system generates a measurement compensation value based on the sensor deviation, and applies the measurement compensation value to the above-ground field characteristics to provide enhanced sensing with above-ground sensors that emulates the accuracy, precision or both of below-ground sensors. Going forward, the system calibrates the measured above-ground field characteristics to establish a correspondence between the below-ground field characteristics and the above-ground field characteristics. Because, in some examples, the below and above ground sensors move together (e.g., are mounted with vehicles or implements) the measurement compensation is readily determined based on sensor deviations as the sensors move through the field.

As a result of calibrating the above-ground field sensor, the system enhances the accuracy, precision, or both of the above-ground field sensing and monitored field characteristics using the above-ground field sensor. In another example, a vehicle (e.g., a sprayer, or the like) that includes above-ground field sensors but does not include below-ground field sensors uses the calibration (e.g., one or more measurement compensation values) determined in previous agricultural operations to enhance the accuracy (or precision) of the measurements of field characteristics (including below-ground characteristics, such as characteristics proximate to the surface of the soil) measured with the above-ground field sensors. Accordingly, the system determines field characteristics without disturbing the soil or requiring instruments that penetrate the soil, for example with crops nearing harvest or with vehicles that do not readily include ground penetrating sensors. Implements and vehicles such as harvesters (combines), sprayers or the like, that do not include below-ground implements optionally includes above-ground sensors and are readily able to provide accurate and precise field characteristic measurements of below and above ground field characteristics that are enhanced with the measurement compensation value (or values) previously determined.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 shows an example of an agricultural vehicle with an agricultural implement, for example a tractor pulling a tiller.

FIG. 2 shows another example of the agricultural implement, for example a planter.

FIG. 3 yet another example of the agricultural implement, for example a cultivator.

FIG. 4 shows still yet another example of the agricultural implement, for example a ripper.

FIG. 5 shows a still yet further example of the agricultural implement, for example a sprayer.

FIG. 6 shows an additional example of an agricultural implement, for example a harvester.

FIG. 7 shows an example of a stationary below-ground field sensor.

FIG. 8 shows another example of the stationary below-ground field sensor, with the sensor located in the field.

FIG. 9A shows an example of field characteristic measurements (e.g., a first set of field characteristics) for an agricultural field indexed to portions of the field where the measurements are taken.

FIG. 9B shows another example of measured field characteristics (e.g., a second set of field characteristics) for the agricultural field 106 indexed to portions of the field where the measurements are taken.

FIG. 10 shows yet another example of measured field characteristics for the agricultural field, for instance after application of a nitrogen-based fertilizer.

FIG. 11 shows still yet another example of measured field characteristics for the agricultural field indexed to portions of the field where the measurements are taken.

FIG. 12 shows an example of a plurality of field thresholds for the agricultural field indexed to portions of the field where the field thresholds are located.

FIG. 13 shows an example of agricultural product applications for the agricultural field indexed to portions of the field where the agricultural product applications are located.

FIG. 14 shows a further example of measured field characteristics for the agricultural field indexed to portions of the field where the measurements are taken.

FIG. 15 shows another illustration of the field with yield values indexed to portions of the field where the yield values were obtained.

FIG. 16A shows a schematic diagram of an example of the field monitoring and husbandry system.

FIG. 16B shows a detailed schematic view of example architecture of the field husbandry controller at the box 16B of FIG. 16A.

FIG. 17 shows a block diagram of an example machine.

### DETAILED DESCRIPTION

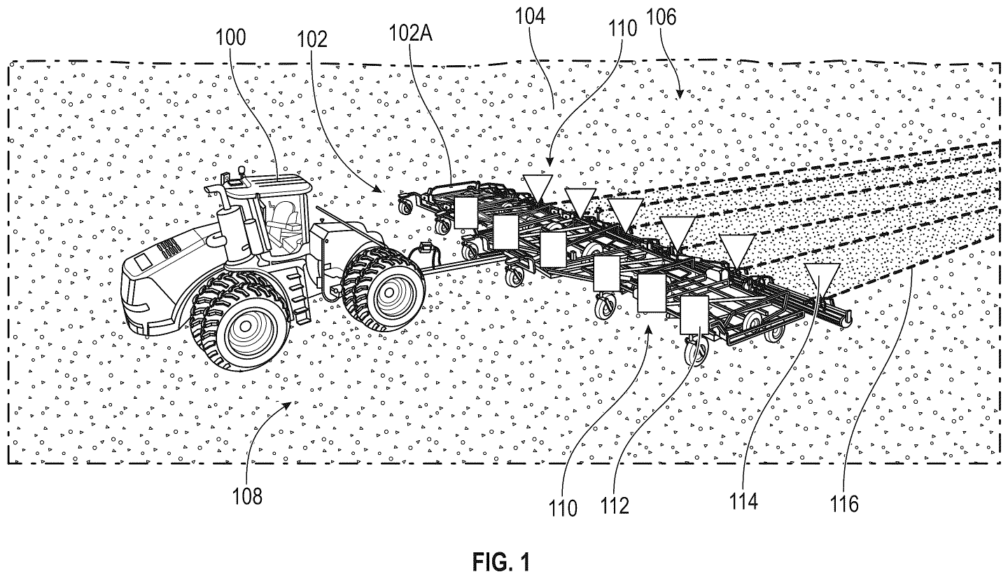

FIG. 1 shows an example of an agricultural vehicle 100, for example a tractor. The vehicle 100 pulls one or more agricultural implements 102, in this example a tiller 102A. The agricultural implement 102A shown tills soil 104 in an agricultural field 106 when towed by the vehicle 100. The one or more agricultural implements includes one or more of the tiller 102A, a plow, a planter, a cultivator, a harvester, a swather, a combine, a sprayer, or a trailer.

In another example, a field monitoring and husbandry system 108 is included in one or more of the vehicle 100 or the agricultural implements 102. The field monitoring and husbandry system 108 shown in FIG. 1 includes one or more field sensors 110. In a further example, the field sensors 110 include one or more of an above-ground field sensor 112 (shown as a rectangle in FIG. 1) or a below-ground field sensor 114 (shown as a triangle in FIG. 1). For instance, one or more of the above-ground field sensors 112 or the below-ground field sensors 114 are mounted with the implement 102 (or the vehicle 100). Accordingly, in some examples, the above-ground field sensors 112 or the below-ground field sensors 114 are on-board (and thereby move with) the implement 102 or the vehicle 100. The above-ground field sensor 112 measures field characteristics, for instance above the surface (e.g., foliage of crops, or the like), at the surface, or below the surface of the soil. The below-ground field sensor 114 measures field characteristics at or below the surface of the soil. For instance, the below-ground field sensor 114 directly contacts (or penetrates) soil. In some approaches, the below-ground field sensor 114 provides greater accuracy or precision of field characteristics relative to the above-ground field sensor 112.

FIG. 1 shows six of the above-ground field sensors 112 mounted to the tiller 102A. Additionally, FIG. 1 shows six of the below-ground field sensors 114 mounted to the tiller 102A. The field sensors 110 (including 112 and 114) measure one or more field characteristics, for example one or more of nitrogen content, phosphorous content, potassium content, ammonia content, ammonium content, moisture content, color of foliage, salinity, pH level, temperature, or the like. In another example, the field sensors 110 measure the field characteristics along at least one measurement path 116 (shown with dashed lines in FIG. 1), such as a row, portion of a swath or the like. Accordingly, the field monitoring and husbandry system 108 measures field characteristics during agricultural operations in the field. In the example shown in FIG. 1, the system 108 measures field characteristics while tilling the field 106.

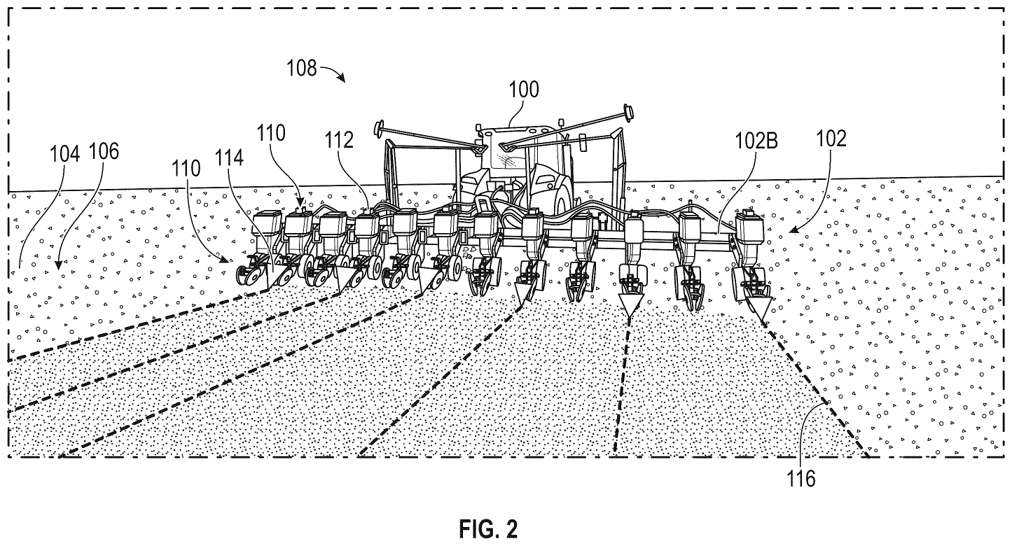

FIG. 2 shows another example of the agricultural vehicle 100, such as a tractor. The vehicle 100 pulls the agricultural implements 102, in this example a planter 102B. The planter 102B plants crops in the agricultural field 106. For instance, the vehicle 100 pulls the planter 102B through the agricultural field 106, and the planter 102B plants the crops in the soil 104 in the manner of a seed drill, seeder or the like. In an example, the implement 102 plants one or more of seeds, seedlings, fruits, vegetables, grasses, legumes, fodder, or the like in the soil 104.

In another example, the field monitoring and husbandry system 108 is included in one or more of the vehicle 100 or the agricultural implements 102 shown in FIG. 2. For instance, the field monitoring and husbandry system 108 includes the field sensors 110. In a further example, the field sensors 110 include one or more of the above-ground field sensors 112 (shown as a rectangle in FIG. 2) or the below-ground field sensors 114 (shown as a triangle in FIG. 2). FIG. 2 shows six of the above-ground field sensors 112 mounted to the planter 102B. Additionally, FIG. 2 shows six of the below-ground field sensors 114 mounted to the planter 102B. In another example, field sensors 110 are provided with each of the row sections of the implement 102 (e.g., corresponding to each seed hopper, disks and wheel assembly) to provide sensing for each of the corresponding rows. The field sensors 110 measure the field characteristics along the at least one measurement path 116 (shown with dashed lines in FIG. 2), such as a row, portion of a swath or the like. Accordingly, in this example the field monitoring and husbandry system 108 measures field characteristics during a planting operation in the field 106.

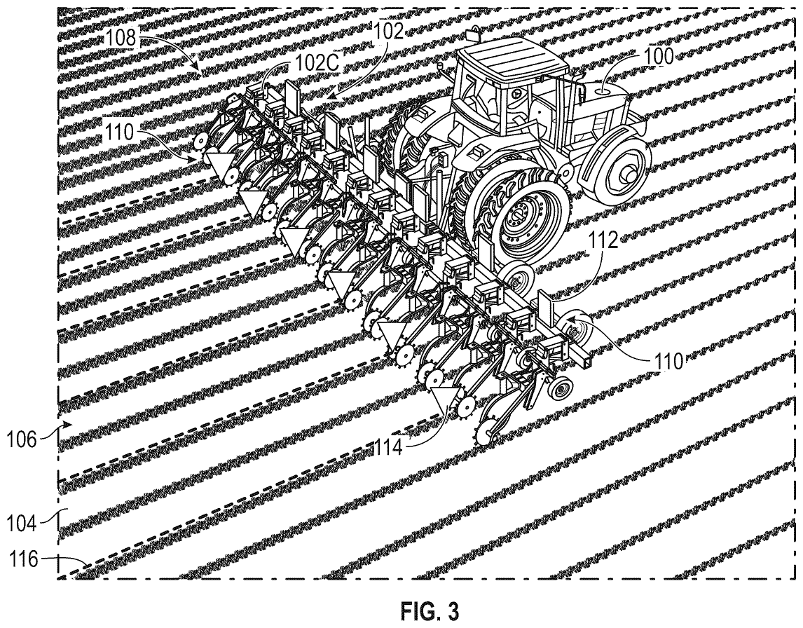

FIG. 3 shows yet another example of the agricultural vehicle 100, for example a tractor. The vehicle 100 pulls the agricultural implements 102, in this example a cultivator 102C. The cultivator 102C shown cultivates the soil 104 in the agricultural field 106 when towed by the vehicle 100. For instance, the implement 102 disturbs (e.g., tills, turns, chops, rips, loosens, or the like) the soil 104 between crops growing in the soil 104. Accordingly, the cultivator manages growth of weeds (and the crops) in the soil 104.

In yet another example, the field monitoring and husbandry system 108 is included in one or more of the vehicle 100 or the agricultural implements 102 shown in FIG. 3. For instance, the field monitoring and husbandry system 108 includes the field sensors 110. In a further example, the field sensors 110 include one or more of the above-ground field sensors 112 (shown as a rectangle in FIG. 3) or the below-ground field sensors 114 (shown as a triangle in FIG. 3). FIG. 3 shows six of the above-ground field sensors 112 mounted to the cultivator 102C. Additionally, FIG. 3 shows six of the below-ground field sensors 114 mounted to the cultivator 102C. The field sensors 110 measure the field characteristics along the at least one measurement path 116 (shown with dashed lines in FIG. 3). Accordingly, in this example, the field monitoring and husbandry system 108 measures field characteristics during cultivation of crops in the field 106.

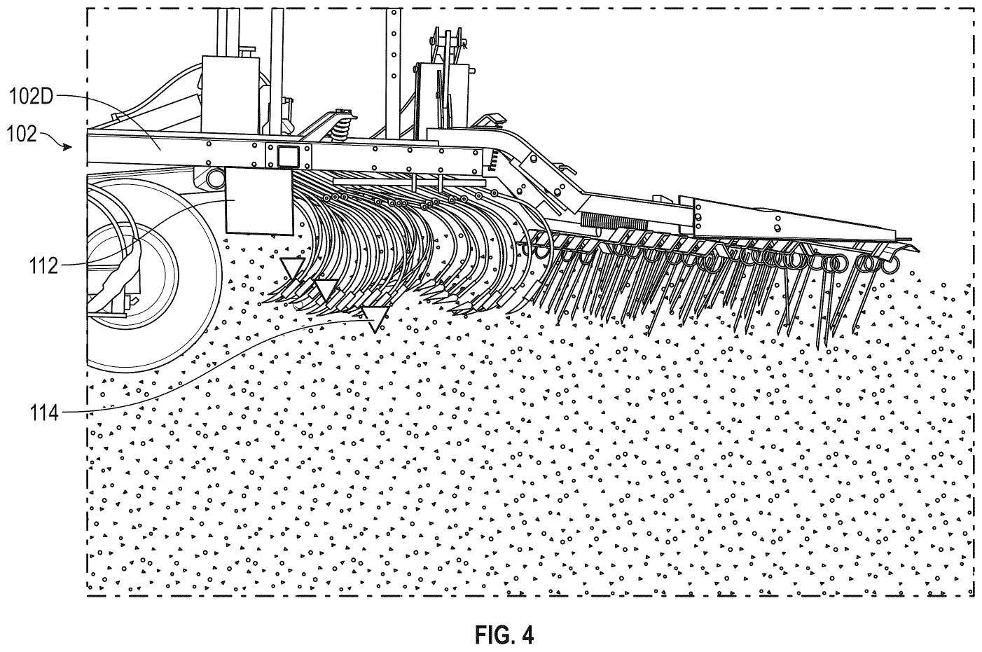

FIG. 4 shows an example of the agricultural implement 102, in this example a ripper 102D. The ripper 102D loosens or aerates the soil 104 in the agricultural field 106. In another example, the vehicle 100 (shown in FIG. 1) tows the ripper 102D through the agricultural field 106, and the ripper 102D disturbs a portion of the soil 104. In yet another example, the field monitoring and husbandry system 108 is included in the agricultural implements 102. In a further example, the field sensors 110 include one or more of the above-ground field sensors 112 (shown as a rectangle in FIG. 4) or the below-ground field sensors 114 (shown as a triangle in FIG. 4).

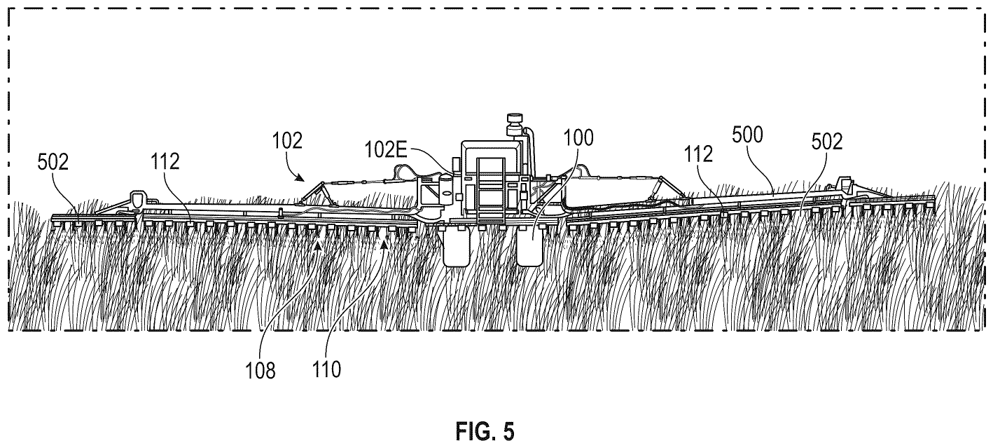

FIG. 5 shows an example of the agricultural implement 102, in this example a sprayer 102E. The sprayer 102E applies an agricultural product to the soil 104 (or crops growing in the soil 104). For instance, the sprayer 102E includes one or more booms 500 with nozzles 502 to spray the agricultural product from the booms 500. In another example, the sprayer 102E is mounted to the vehicle 100, such as a tractor.

In yet another example, the field monitoring and husbandry system 108 is included in the sprayer 102E. In a further example, the field sensors 110 include one or more of the above-ground field sensors 112 (shown as a rectangle in FIG. 5). In some examples, for instance during spraying of agricultural product, the implement 102 does not disturb the soil 104 (shown in FIG. 1). Accordingly, the system 108 uses one or more of the above-ground field sensors 112 to measure above-ground field characteristics, for instance during spraying of agricultural product. Thus, the field monitoring and husbandry system 108 uses different combinations of field sensors 110 based on the implement 102 moving through the agricultural field 106. For example, the system 108 refrains from including the below-ground field sensor 114 (shown in FIG. 1) during spraying to minimize disturbance to the soil 104 (shown in FIG. 1).

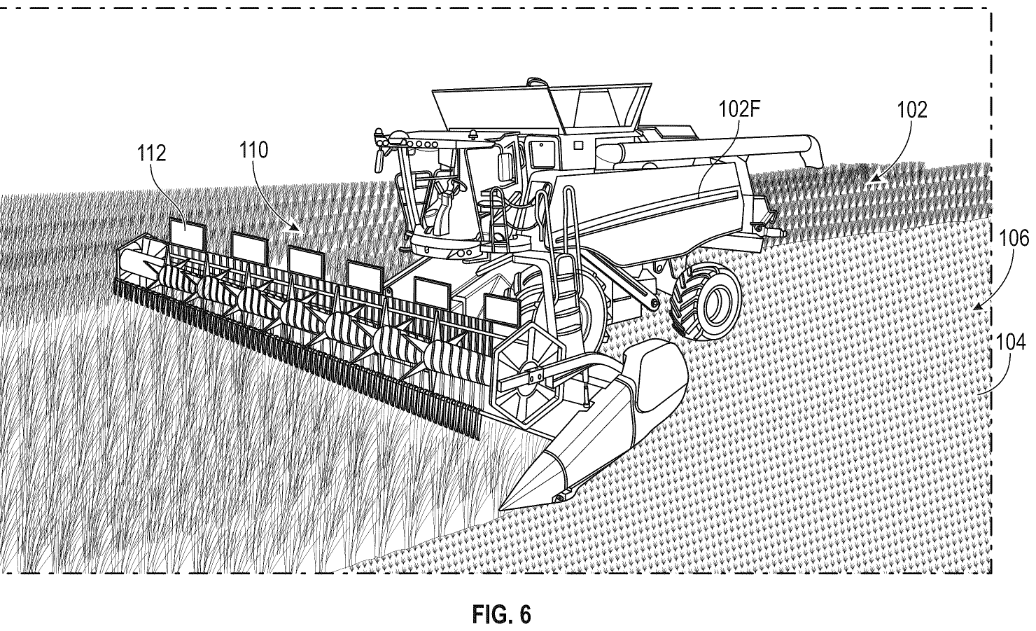

FIG. 6 shows an example of the agricultural implement 102, in this example a harvester 102F. The harvester 102F harvests crops grown in the agricultural field 106. In yet another example, the field monitoring and husbandry system 108 is included in the agricultural implements 102. In a further example, the field sensors 110 include one or more of the above-ground field sensors 112 (shown as a rectangle in FIG. 6). The field monitoring and husbandry system 108 uses one or more of the above-ground field sensor 112 to measure above-ground field characteristics, for instance during harvesting of crops.

The field monitoring and husbandry system 108 uses different combinations of field sensors 110 (based on the implement 102) that monitor field characteristics as the implement moves through the agricultural field 106. For example, the system 108 uses below-ground field sensors during preparation of the field 106 for growing of crops. With crops growing in the field 106, the system 108 optionally refrains from including the below-ground field sensor 114 (shown in FIG. 1) to minimize disturbance to the soil 104. Accordingly, the system 108 uses one or more of the above-ground field sensors 112 to measure field characteristics in correspondence with minimizing disturbance to the soil 104. Alternatively, the below-ground field sensors 114 are included with implement tools that are used between crop rows (e.g., cultivator blades).

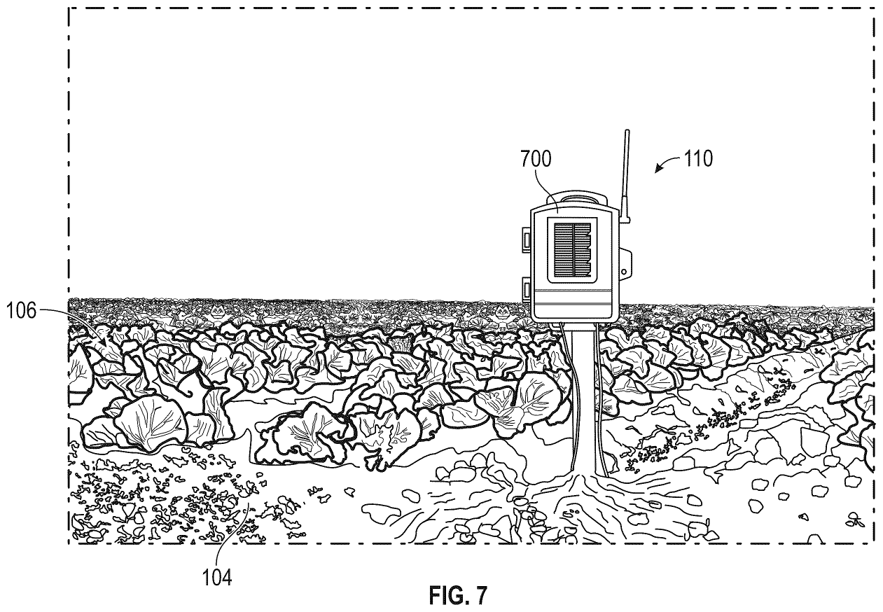

FIG. 7 shows an example of a stationary below-ground field sensor 700. In some examples, the stationary below-ground field sensor 700 is stationed in the agricultural field 106. For instance, the below-ground field sensor 700 is not mounted with the agricultural implements 102 or the vehicle 100. Accordingly, the below-ground field sensor 700 measures below-ground field characteristics in a single location (or proximate the single location) in the field 106. In contrast, the below-ground field sensor 114 mounted with (or on-board) the vehicle 100 or the implement 102 (shown in FIG. 1) measures below-ground field characteristics throughout the field 106 (optionally including proximate to the single location where the stationary below-ground field sensor 700 is located in the field 106).

In an example, the field sensors 110 include one or more of a laser sensor, hyperspectral sensor, multispectral sensor, NDVI sensor, satellite sensor, ultrasonic sensor, ground penetrating radar sensor, gamma ray sensor, infrared sensor, temperature sensor, moisture sensor, pH sensor, or the like. For instance, the field sensors 110 include a transducer, for instance an electromagnetic emitter and an electromagnetic receiver. In another example, the system 108 uses satellite imagery as a sensor input. For instance, the above-ground field sensors 112 include a sensor included in a satellite (e.g., a satellite orbiting Earth, or the like). The system 108 optionally uses the satellite-based sensor to refine field characteristics measured with the on-board field sensors 110 or the stationary below-ground field sensor 700. For example, the system 108 downloads a satellite image and uses the satellite image to make a determination on the field characteristics. The system 108 optionally marries the satellite imagery with the measurements provided by the on-board field sensors 110 or the stationary below-ground field sensor 700. For instance, the satellite image indicates an NDVI difference in part of the field 106. The NDVI difference, in some examples, is due to lack of moisture. In other examples, the NDVI difference is due to lack of nutrients. The system 108 uses the on-board field sensors 110 or the stationary below-ground field sensor 700 to understand the NDVI difference in the satellite imagery.

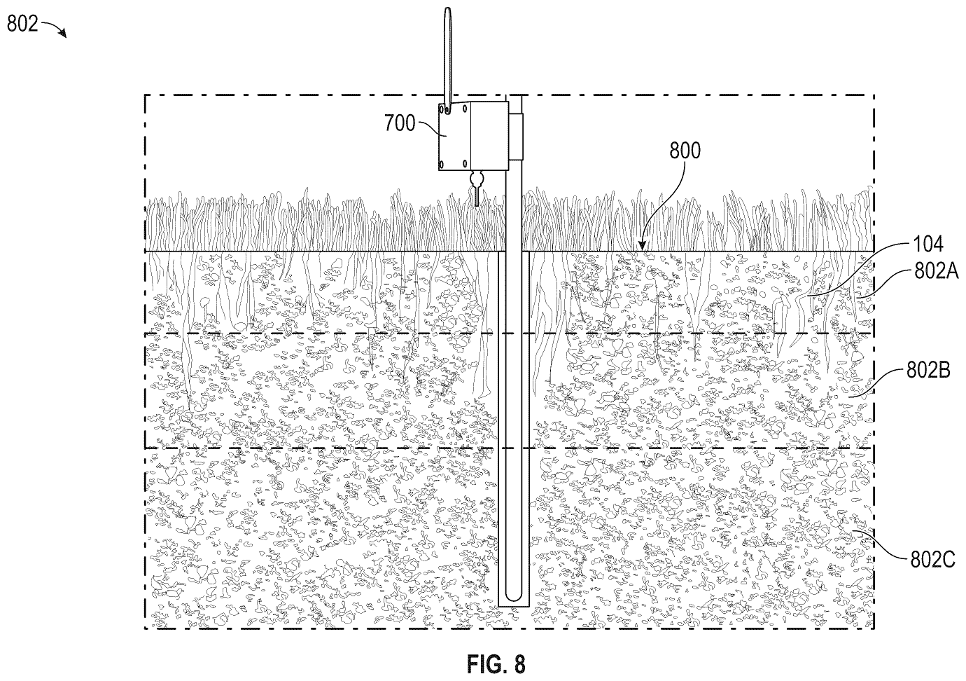

FIG. 8 shows another example of the stationary below-ground field sensor 700, with the sensor 700 located in the field 106. Additionally, FIG. 8 shows the below-ground field sensor 700 extending below a surface 800 of the soil 104. In an example, the below-ground field sensor 700 measures below-ground field characteristics at one or more layers 802 in the soil 104. For instance, the below-ground field sensor 700 measures below-ground field characteristics at one or more of a first layer 802A, a second layer 802B, or a third layer 802C. In an example, the below-ground field characteristics vary based on the layers 802 in the soil 104. For example, crops growing in the soil 104 have roots 804 that extend between the layers 802. Accordingly, measuring of the below-ground field characteristics across the layers 802 helps determine which field characteristics the crops are exposed to during a growth season.

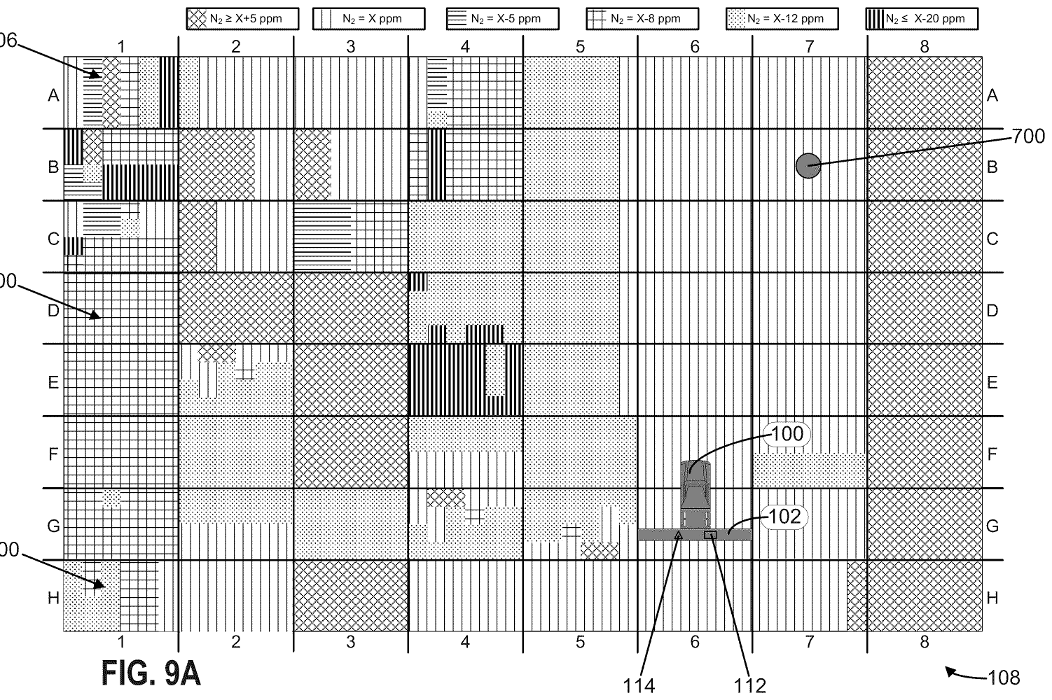

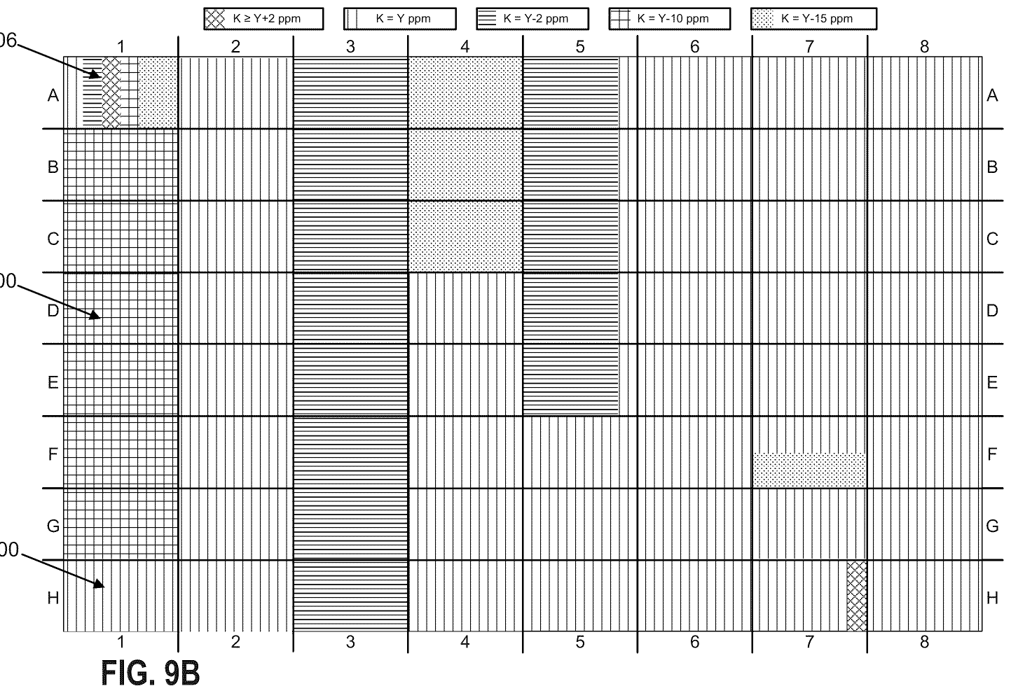

FIG. 9A shows an example of field characteristic measurements for the agricultural field 106 indexed to portions of the field where the measurements are taken. For instance, FIG. 9A shows nitrogen content as the measured field characteristic—however the present subject matter is not so limited. In an example, the field monitoring and husbandry system 108 (e.g., with the sensors 110 shown herein) measures field characteristics including one or more of nitrogen content, phosphorous content, potassium content, ammonia content, ammonium content, moisture content, color of foliage, salinity, pH level, temperature for the agricultural field 106. In one example, the field characteristics shown in FIG. 9A are measured before a growing season—for example with a tiller (e.g., in the spring or the fall) as the agricultural implements 102 having field sensors 110, such as below-ground field sensors 114, navigates the field 106.

FIG. 9A shows the agricultural field 106 having a plurality of zones 900. For example, the zones 900 include zone 1A-H, 2A-2H, 3A-3H, 4A-4H, 5A-5H, 6A-6H, 7A-7H, and 8A-8H (also referred to as “zones 1A-8H”). The system 108 measures the field characteristics within the zones 900 (or with potentially greater resolution within portions of the zones 900, sub-zones, rows, portions of swaths, or the like). Providing multiple sensors 110 facilitates corresponding greater resolution of sensing. In one example of higher resolution measurement, the nitrogen content varies within zone 1A as shown in FIG. 9A. FIG. 9A further shows zone 1A having a nitrogen content of less than or equal to X−20 parts per million in a first portion, and greater than or equal to X+5 parts per million in a second portion (with X being a specified content of nitrogen). The nitrogen content is expressed in units of parts per million (“ppm”)—however the present subject matter is not so limited.

Zone 1A is an illustrative example of the application of agricultural products in varying concentrations—with the resulting field characteristics shown in FIG. 9A after those applications. The variations shown in in zone 1A are provided to indicate a potential resolution of the measured field characteristics. The variations shown in zone 1A are exemplary, and provided to assist reader comprehension of FIG. 9A. For instance, varying concentrations of agricultural product are applied within zone 1A, and the nitrogen content is measured to assess how the varying quantities of agricultural product affect the nitrogen content within zone 1A. Accordingly, the system 108 measures varying amounts of nitrogen content across zone 1A (as well as the other zones). In contrast, the nitrogen content is constant throughout zone 6A (as an example) indicating nitrogen in the zone is generally consistent throughout zone 6A. In another example, FIG. 9A shows the nitrogen content is lower in zone 4E than the nitrogen content of zone 6A.

In yet another example, FIG. 9A shows the vehicle 100 with implement 102 (in zones 6F, 6G) navigating through the agricultural field 106. In this example, the implement has one or more above-ground field sensors 112 and one or more below-ground field sensors 114 (collectively field sensors 110). In some approaches, the field sensors 110 mounted with the implement 102 (or vehicle 100), for instance the above-ground field sensors 112 have limited accuracy, precision or both for sensing characteristics below ground in comparison to stationary below-ground field sensor 700. In an example, the stationary below-ground field sensor 700 is installed in the field (e.g., driven into the soil) and measures field characteristics at or below the surface of the soil. For instance, the stationary below-ground field sensor 700 directly contacts soil and provides greater accuracy or precision of field characteristics relative to the above-ground field sensor 112.

However, the stationary below-ground field sensor conducts measurements at its location in the field and does not provide measurements throughout the field 106. In contrast field sensors 110 mounted with the implement 102 or vehicle 100 move with the vehicle and accordingly conduct measurements throughout the field, wherever the vehicle and implement travel. The above-ground field sensors 112 (and below-ground field sensors 114 with the vehicle or implement) sense field characteristics in correspondence with the vehicle 100 navigating the field. As noted herein the measured field characteristics are readily indexed to the associated portions of the field. Accordingly, the above-ground field sensors 114 and below-ground field sensors 112 (mounted with the vehicle or implement) facilitate enhanced indexing of measurements of field characteristics to portions of the field and thereby provide higher resolution measurement of characteristics through the field in comparison to static sensors, such as the stationary below-ground sensor 700.

In some examples, the field monitoring and husbandry system 108 uses measurements from one or both of the above-ground field sensors 112 or the below-ground field sensors 114 mounted with the implement 102 (or the vehicle 100) to determine the field characteristics, for example nitrogen content. In another example, the system 108 uses measurements from the stationary below-ground field sensor 700 to determine the field characteristics. For instance, the system 108 assigns weight to measurements from the above-ground field sensors 112 or the below-ground field sensors 114 mounted with the implement 102 (or the vehicle 100) based on proximity of the (on-board) sensors 112, 114 to the stationary below-ground field sensor 700. In an example, the stationary below-ground field sensor 700 has enhanced accuracy or precision (or both) in comparison to one or more of the above-ground field sensors 112 or the below-ground field sensors 114 mounted with the implement 102. Accordingly, the system 108 uses measurements obtained from the stationary below-ground field sensor 700 based on proximity of the implement 102 (having the above-ground field sensors 112 or the below-ground field sensors 114) in relation to the stationary below-ground field sensor 700. For instance, with the vehicle 100 or implement 102 near to the stationary below-ground field sensor 700 (e.g., within one swath, within 5 feet, 10 feet, 25 feet or so on) the system 108 weights the stationary sensor 700 measurements higher. Conversely, with the vehicle 100 or implement relatively remote to the field sensor 700 (e.g., beyond one swath, greater than 50 feet, 100 feet or the like) the measurements of the sensors 112, 114 are provided a greater weight. Thus, in this example the accuracy or precision (or both) of measured field characteristics is enhanced by weighting of the measurements provided by the field sensors 110 based on proximity to the stationary below-ground field sensor 700.

FIG. 9B shows another example of a measured field characteristic for the agricultural field 106 indexed to portions of the field where the measurements are taken. For instance, FIG. 9B shows potassium content as the measured field characteristic—however the present subject matter is not so limited. In one example, the field characteristics shown in FIG. 9B are measured before a growing season—for example with a tiller (e.g., in the spring or in the fall) as the agricultural implements 102 having field sensors 110, such as below-ground field sensors 114, navigates the field 106. In another example, the potassium content (shown in FIG. 9B) is measured contemporaneously with the measurement of the nitrogen content (shown in FIG. 9A), for instance with the same implement/sensor. Accordingly, in an example, the system 108 measures two or more field characteristics for each associated portion of the plurality of associated portions (e.g., for each zone of the plurality of the zones 900, or the like).

FIG. 9B shows the agricultural field 106 having the plurality of zones 900. The system 108 measures the field characteristics within the zones 900 (or with potentially greater resolution within portions of the zones 900, sub-zones, rows, portions of swaths, or the like). In one example of higher resolution measurement , the potassium content varies within zone 1A. FIG. 9B shows zone 1A having a potassium content between Y−15 parts per million and greater than or equal to Y+2 parts per million (with Y being a specified content of nitrogen).

In another example, zone 1A is used as a test bed for application of agricultural product to change the potassium content within zone 1A. For instance, varying quantities of agricultural product are applied within zone 1A, and the potassium content is measured to assess how the varying quantities of agricultural product affect the potassium content within zone 1A. Accordingly, the system 108 measures varying amounts of potassium content across zone 1A. In contrast, the potassium content is constant throughout zone 6A. In another example, FIG. 9B shows the potassium content is lower in zone 4A than the potassium content of zone 6A

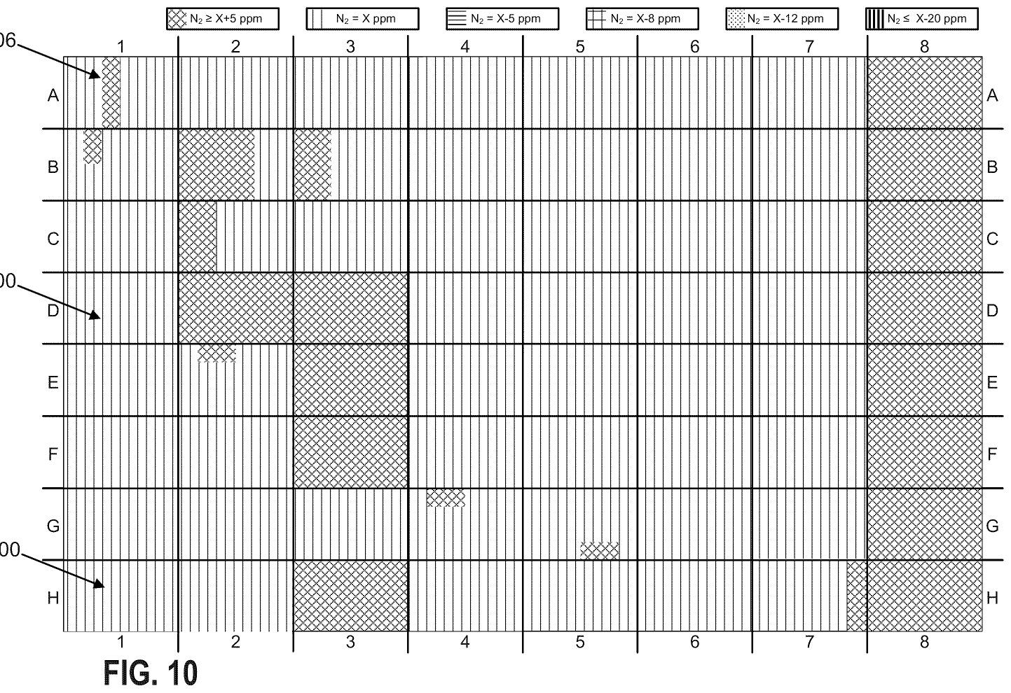

FIG. 10 shows another example of measured field characteristics for the agricultural field 106, for instance after application of a nitrogen-based fertilizer. In this example, nitrogen content is the measured field characteristic—however the present subject matter is not so limited. The field characteristics shown in FIG. 10 are from measurements conducted during a growing season and after a nitrogen application—for example with a planter as the agricultural implement with above-ground field sensors 112 performing the measurements. The agricultural products have been applied previously to adjust field characteristics, for instance to promote yield, crop health or the like. Thus, the field characteristics are adjusted using the field monitoring and husbandry system 108 thereby achieving the (updated) field characteristics shown in FIG. 10. For example, the system 108 has previously determined the field characteristics (e.g., shown in FIG. 9A) and has applied agricultural product (or products) to achieve the field characteristics shown in FIG. 10. FIG. 10 shows the field characteristics are relatively consistent, and accordingly the system 108 addresses deficiencies noted in FIG. 9A.

In another example, the system 108 changes the field soil in the agricultural field 106 to achieve a minimum (prescribed) value for nitrogen content in the zones 900. FIG. 10 is a sensor-based confirmation of at least a minimum prescribed value for nitrogen content is achieved with the preceding application. For instance, FIG. 10 shows the zones 900 having X ppm or X+5 ppm of nitrogen. Accordingly, FIG. 10 shows the system 108 changed the field soil in the zones 900 that previously had nitrogen content less than X ppm. For instance, the system 108 uses the sprayer 102E to change the field characteristics before planting of crops in the agricultural field 106 (and after measurement of the field characteristics before the season, shown in FIG. 9A). In yet another example, zone 5F had a nitrogen content of X−12 ppm at the beginning of the season (shown in FIG. 9A). The system 108 applied agricultural product to zone 5F to achieve the prescribed value (in this example, X ppm) for nitrogen content in zone 5F (shown in FIG. 10). In still yet another example, zone 6A had the prescribed value (X ppm, shown in FIG. 9A) of nitrogen content at the beginning of the season. Similarly, FIG. 9A shows zone 8A had a nitrogen content (X+5 ppm) that was greater than the prescribed value (X ppm). Accordingly, the system 108 refrained from changing the field soil (with agricultural product) in zones 6A and 8A prior to planting of crops. Thus, in this example, the system 108 refrains from conditioning of the soil in some zones as the nitrogen content in zones 6A and 8A remain the same between the beginning of the season (shown in FIG. 9A) and during planting of crops in the field (shown in FIG. 10). Consequently, in other zones the system 108 measures one or more field characteristics (in this example, nitrogen content), determines deviations and applies husbandry to the agricultural field 106 based on the measured field characteristics and deviations from specified thresholds to achieve prescribed values for the field characteristics.

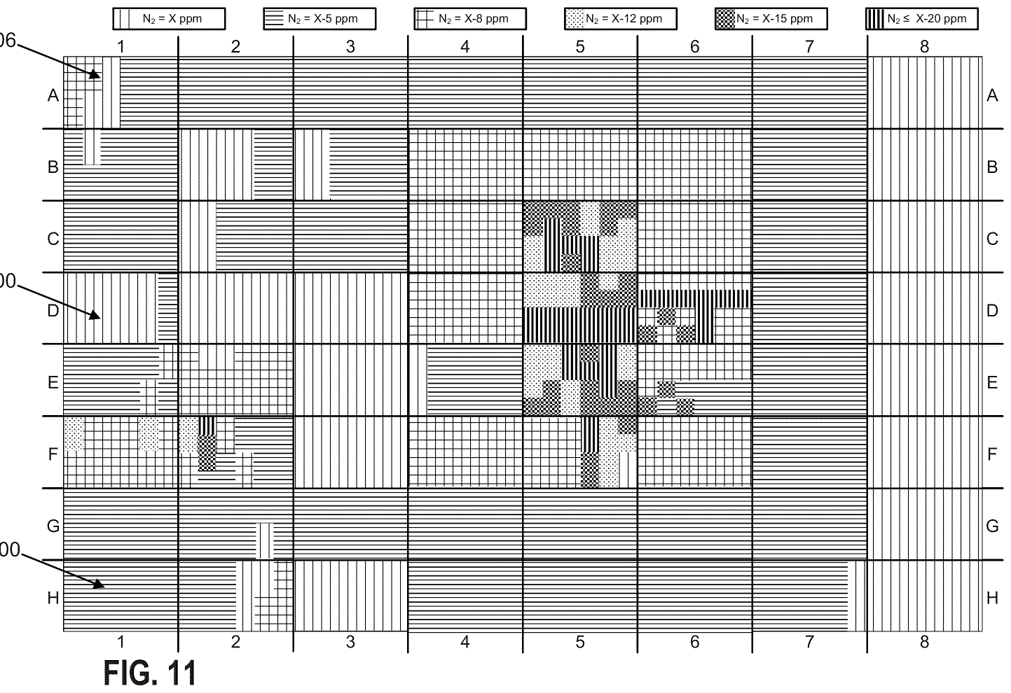

FIG. 11 shows still yet another example of measured field characteristics for the agricultural field 106 indexed to portions of the field where the measurements are taken. For instance, FIG. 11 shows nitrogen content as the measured field characteristic—however the present subject matter is not so limited. In one example, the field characteristics shown in FIG. 11 are measured during a growing season, for instance after planting of crops and during growth of the crops in the agricultural field 106 and after the measurements reflected in FIG. 10. For example, the cultivator 102C (an example agricultural implement 102, shown in FIG. 3) is navigated through the field 106, and one or more of above-ground 112 or below-ground field sensors 114 measure field characteristics during cultivation of the field 106. Accordingly, the system 108 measures field characteristics while crops are growing in the field 106.

The field monitoring and husbandry system 108 uses the measured field characteristics shown in FIG. 11 to enhance growth, yield, or the like of the crops in the field. In an example, the field characteristics change as crops grow in the field 106. For instance, the crops growing in the field use nutrients (e.g., nitrogen, or the like) from the field soil. Accordingly, in this example, the content of the nutrients in the field soil decreases as the crops grow in the field 106. For instance, the nitrogen content of the field is depleted by crops growing in the field.

The system 108 measures the field characteristics, and readily determines changes in field characteristics while crops grow in the field 106. In an example, FIG. 11 shows the nitrogen content has decreased (in comparison to the nitrogen content shown in FIG. 10) in portions of the field 106, for instance in zones 5C, 5D, and 5E. Additionally, FIG. 11 shows the zones 5C, 5D, 5E have decreased at a greater rate in comparison to other portions (e.g., zones 4B, 4C, 4D, 6B, 6C) of the field 106 while crops are growing in the field 106. For example, FIG. 10 shows zones 5C, 5D, 5E had the same nitrogen content as other portions (e.g., zones 4B, 4C, 4D, 6B, 6C) of the field 106. However, during growth of the crops, FIG. 11 shows zones 5C, 5D, 5E having nitrogen content decreasing at a greater rate than other portions of the field 106. Accordingly, the field monitoring and husbandry system 108 readily identifies portions of the field 106 that deplete nutrients or the like quickly, and correspondingly have a greater need for supplementation of (in this example) nitrogen content. In an example, the system 108 uses the measured characteristics shown in FIG. 11 and specified thresholds for those characteristics, determines deviations therebetween and then conditions the field characteristics, for instance with application of agricultural products (e.g., having nitrogen, other nutrients, or the like) to enhance nitrogen content in the agricultural field 106.

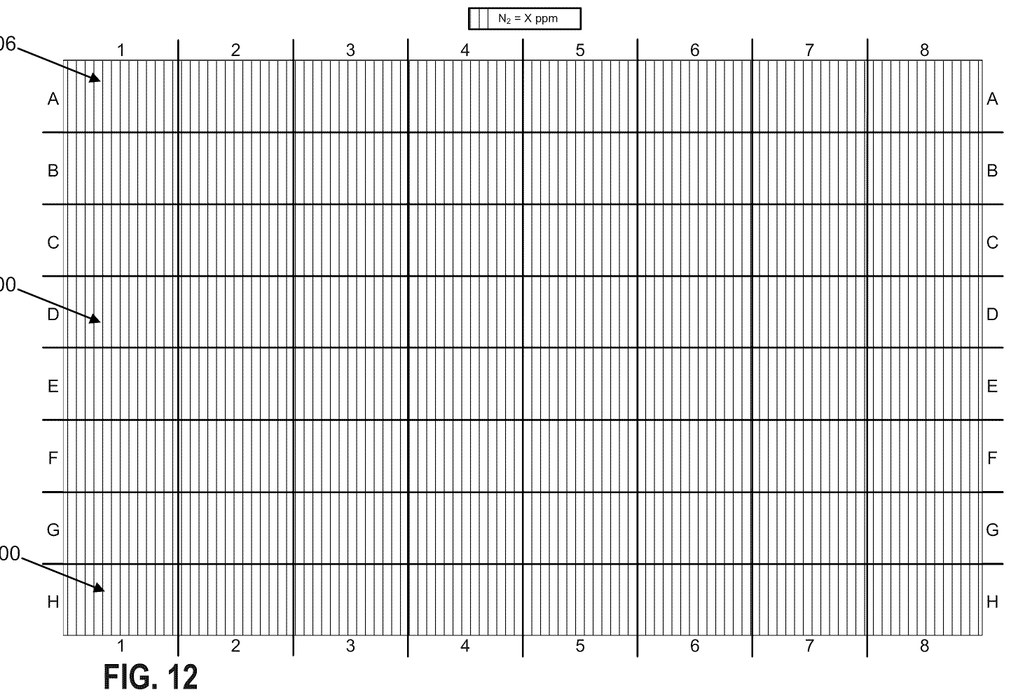

FIG. 12 shows an example of a plurality of field thresholds for the agricultural field 106 indexed to portions of the field where the field thresholds are located. In an example, the field thresholds are specified values (e.g., target values, prescribed values, or the like) for the field characteristics. In another example, the field threshold is indexed to a corresponding portion of the agricultural field 106. For instance, the field thresholds shown in FIG. 12 are X ppm of nitrogen content for all of the zones 900 in the field 106 (e.g., each of zones 1A-8H have a field threshold, also referred to as a target value or prescription, of X ppm). In an example, the field monitoring and husbandry system 108 uses the field threshold as a target value for the field characteristic within the corresponding portion of the field. The system 108 optionally applies an agricultural product to the agricultural field 106 to achieve the target value (field threshold) of the characteristic nutrient, constituent or the like indexed to the zones 900. For example, the previous measurements of field characteristics (e.g., as in FIG. 11) are ‘current’ values, the field thresholds are target values, and the deviation therebetween is representative of the quantity of agricultural product for application to achieve the specified field threshold. Accordingly, the system 108 treats (changes) the characteristics of the soil based on the measured characteristics to achieve the field threshold for the zones 900 in the field 106.

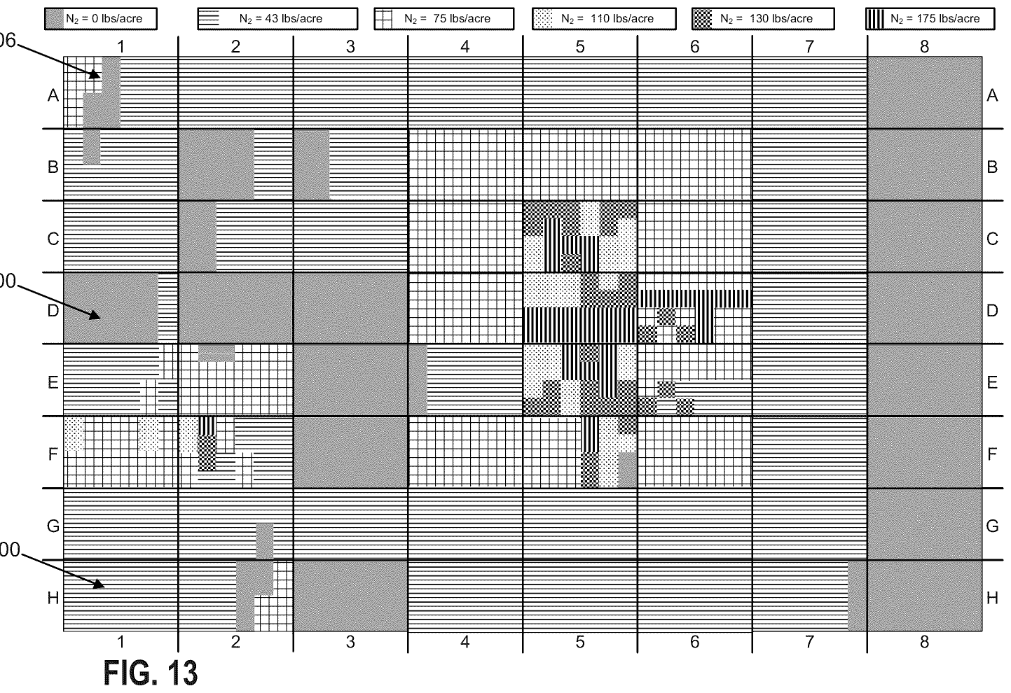

FIG. 13 shows an example of agricultural product applications for the agricultural field 106 indexed to portions of the field where the agricultural product applications are located. The agricultural product applications are configured to meet or achieve the field thresholds (such as the field thresholds shown in FIG. 12). In an example, the field monitoring and husbandry system 108 generates an application rate, and the system 108 applies the agricultural product according to the application rate. For instance, the system 108 compares the measured field characteristic(s) (e.g., nitrogen content after planting of crops, shown in FIG. 11) to the one or more field thresholds (e.g., specified or target value of nitrogen content of X ppm in FIG. 12). The system 108 determines deviations between the field thresholds and the measured field characteristics, and selects application rates based on the deviations as shown with the varied hatching in FIG. 13. Accordingly, the system 108 generates application rates based on the comparison of the measured field characteristic(s) with one or more field thresholds. When applied at the illustrated application rates, the agricultural product guides the soil characteristic toward the specified value (e.g., the field threshold, a prescription value, or the like) thereby decreasing the deviation.

In an example, FIG. 12 shows zones 8A-8H have an application rate of zero. The application rate is zero in zones 8A-8H because the nitrogen content in zones 8A-8H was X ppm when the field characteristics were measured (shown in FIG. 11) and the field threshold for zones 8A-8H is X ppm. Accordingly, the deviations between the field threshold and the measured field characteristics is zero (including approximately zero) and the system 108 refrains from applying agricultural product in zones 8A-8H.

In another example, FIG. 12 shows zones 7A-7G have a consistent application rate of 43 pounds of nitrogen per acre. The system 108 determines the application rate by comparing the measured nitrogen content in zones 7A-7G (e.g., X−5 ppm, shown in FIG. 11) with the field threshold for zones 7A-7G (e.g., X ppm, shown in FIG. 12) and determining a deviation therebetween. The system 108, look up table or the like equates an application rate of agricultural product to the determined deviation and prescribes that application rate for application. Thus, the system 108 applies the agricultural product at 43 pounds of nitrogen per acre to change (control) the soil in zones 7A-7G and guide the field characteristics toward the specified field threshold, also referred to as one or more specified values of field characteristics. The field monitoring and husbandry system 108 thereby enhances correspondence (e.g., decreases deviation) between the measured field characteristics and one or more specified values of field characteristics by applying the prescribed agricultural products to the field based on the prescriptions and thereby changing the field characteristics to the specified values.

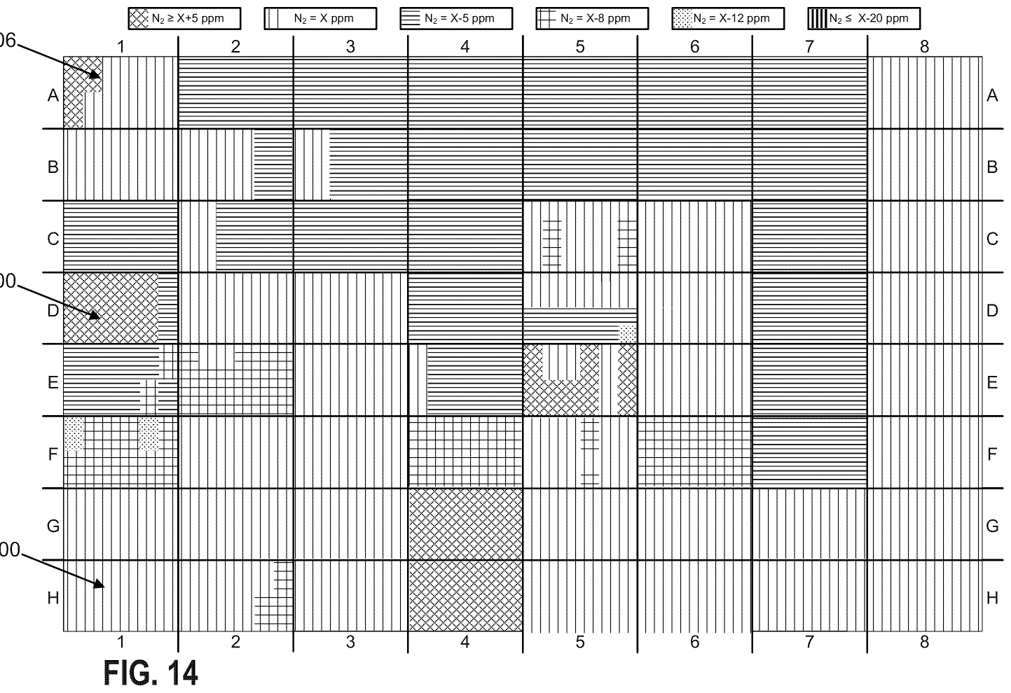

FIG. 14 shows a further example of measured field characteristics for the agricultural field 106 indexed to portions of the field where the measurements are taken. For instance, FIG. 14 shows nitrogen content as the measured field characteristic—however the present subject matter is not so limited. In one example, the field characteristics shown in FIG. 14 are measured during harvest of crops in the field 106. For example, the harvester 102F (an example agricultural implement 102, shown in FIG. 6) is navigated through the field 106, and above-ground field sensors 112 measure field characteristics during harvesting of crops from the field 106 and before planting of the crops in the next season.

Accordingly, the system 108 measures field characteristics at different times, for instance before planting of crops (e.g., in fall or spring, shown in FIGS. 9A and 9B), while crops are growing (e.g., shown in FIG. 11), and during (or after) harvesting of crops from the field 106 (e.g., shown in FIG. 14). For example, the system 108 monitors (or logs) the field characteristics for a portion of the field 106 and associates the monitored field characteristics with the yield value from that portion of the field 106.

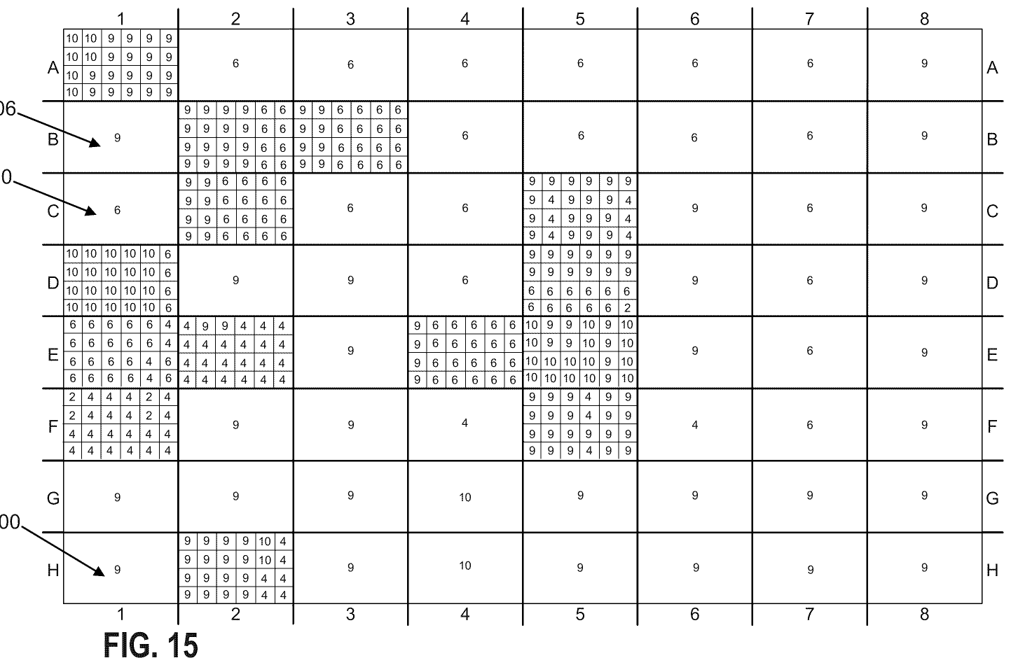

FIG. 15 shows another illustration of the field 106. In this example, yield values are indexed to zones of the field. In some examples, the system 108 generates a map of the field 106 including the yield values indexed to the zones of the field 106. Optionally, yield values are further indexed in some of the zones at a higher resolution, for instance sub-zones of the zones. In an example, the field monitoring and husbandry system 108 includes a yield monitor (e.g., as a component of a harvester or combine), and the yield monitor measures one or more yield values for the crops grown in the field 106.

As shown in FIG. 15, the yield values are indexed to associated portions of the agricultural field 106. For example, FIG. 15 shows the yield value (ranging between one and ten) for each zone (or sub-zone) within the agricultural field 106. In one example, the yield value is quantitative, for instance indicating the quantity of crops harvested from a zone (or sub-zone). In an example, a zone having a yield value of ten indicates a mass (e.g., weight, bulk, or the like) of crops measured in that zone is ten times that of a different zone having a yield value of one. In another example, the yield value is qualitative, for instance indicating the quality (e.g., moisture content, or the like) of crops harvested from a zone (or sub-zone). In still other examples, the yield value is a multi-component value including both quantity, moisture content, or is a refined quantity value adjusted for moisture content.

In an example, zone 2A has a yield value of six, and zone 4H has a (greater) yield value of ten. Accordingly, FIG. 15 shows the yield value for zones 1B and 4H are constant throughout those zones (and only shows the yield value once within each of those zones). In contrast, zone 1A has varied yield values. Thus, the yield values for each sub-zone within zone 1A are shown in FIG. 15. For instance, FIG. 15 shows zone 1A has sub-zones with 24 yield values (in this example, values of nine or ten).

In another example, the yield values (shown in FIG. 15) are associated with the measured field characteristics (shown in one or more of FIG. 9, 10, 11, or 12). For instance, the field monitoring and husbandry system 108 associates the yield values for the zones 900 with one or more field characteristic measurements of the zones 900 measured with agricultural implements (e.g., the implement 102 shown in FIG. 1, or the like). For example, the yield value of zone 8A (e.g., nine, shown in FIG. 15) is associated with the field characteristics of zone 8A (e.g., X ppm of nitrogen content, shown in FIG. 14 and optionally other measurements illustrated in Figures herein). Accordingly, the system 108 measures one or more field characteristics over the course of one or more seasons, and associates the yield value for crops with the measured field characteristics. Thus, the system 108 monitors the yield values, and associates the yield values with field characteristics measured in the same zones, sub-zones or the like. As discussed herein, the relationships between yield, associated measured field characteristics including changes in measured field characteristics (e.g., depletion) enhance the prescribed application of agricultural products to enhance yield.

In an embodiment, the system 108 monitors the quantity of agricultural product(s) applied to the field 106. For instance, the system 108 logs (e.g., records, meters, quantifies, monitors, or the like) the quantity of agricultural product applied to a zone, and (as discussed herein) measures field characteristics of the zone based on the application of the agricultural product to the zone including changes in field characteristics after application relative to preceding measurements of the field characteristics. Consequently, the system 108 monitors one or more of the field characteristics, the agricultural product applied to the field 106, or the yield values for crops grown in the field 106. As discussed herein, the system 108 uses combinations of the monitored field characteristics, the agricultural product applied to the field 106, or the yield values for crops grown in the field 106 to inform future husbandry of the agricultural field 106.

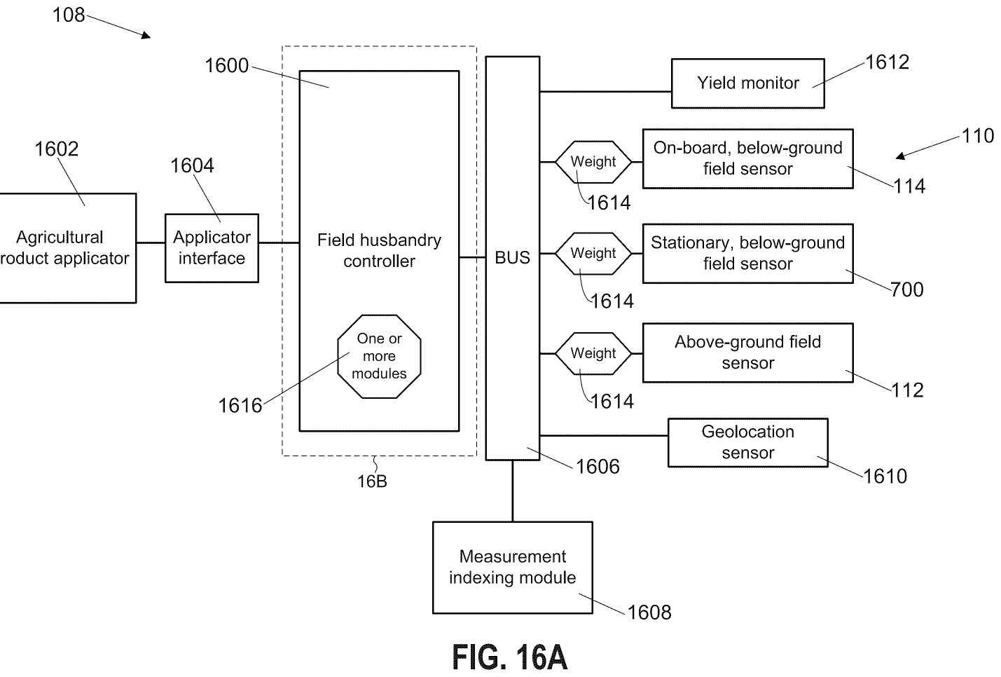

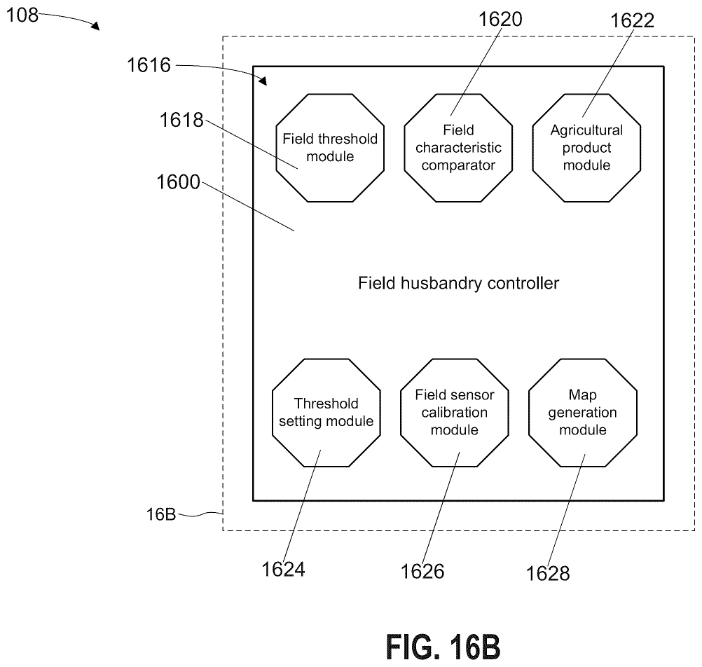

FIG. 16A shows a schematic diagram of an example of the field monitoring and husbandry system 108. In an example, the system 108 includes a field husbandry controller 1600, such as a field computer, field computer with a husbandry module, dedicated husbandry controller or the like. The field husbandry controller 1600 cooperates with other components of the system 108 to monitor field characteristics of the agricultural field 106 (shown in FIG. 1). In another example, the field husbandry controller 1600 cooperates with other components of the system 108 (e.g., the agricultural product application 1602 such as a sprayer, spreader or the like) to apply agricultural product to the agricultural field 106 (shown in FIG. 1).

For example, the system 108 includes an agricultural product applicator 1602. The system 108 operates the agricultural product applicator 1602 using the field husbandry controller 1600 interconnected with an applicator interface 1604. For instance, the field husbandry controller 1600 uses the applicator interface 1604 in the manner of electronic control unit (ECU) to operate the agricultural product applicator 1602. In another example, the applicator interface 1604 includes a relay, and the field husbandry controller 1600 uses the relay to operate the agricultural product applicator 1602. Accordingly, in an example, the applicator interface 1604 facilitates communication between the field husbandry controller 1600 and the agricultural product applicator 1602, and optionally interprets information from the field husbandry controller 1600, such as prescriptions or deviations between thresholds and measured field characteristics, to conduct application with the applicator 1602. In other examples, the field husbandry controller 1600 generates one or more application rates (e.g., based on a prescription, deviations as discussed herein, or the like), and the field husbandry controller 1600 uses the applicator interface 1604 as a relay to the agricultural product applicator 1602 to apply agricultural product at specified application rates.

The agricultural product applicator 1602 communicates with a reservoir (e.g., a storage tank, pit, or the like) and receives agricultural product (e.g., fertilizer, herbicide, insecticide, slurry, or the like) from the reservoir. In an example, the agricultural product applicator 1602 includes the nozzles 502 (shown in FIG. 5), and the agricultural product applicator 1602 sprays the agricultural product onto crops using the nozzles 502 according to application rates provided by the field husbandry controller 1600. In other examples, the applicator 1602 includes a spreader having spreading wheels, a slurry applicator or the like.

FIG. 16A shows a bus 1606 that facilitates communication between components of the field monitoring and husbandry system 108. In an example, the field husbandry controller 1600 communicates with field sensors 110 using the bus 1606. In various examples, the sensors 110 include, but are not limited to, above-ground field sensors 112, below-ground field sensors 114, stationary below-ground field sensors 700, or geolocation sensors 1610 (e.g., GPS, real time kinematics or the like). In another example, the field sensors 110 include a yield monitor 1612, for instance provided with a harvester, grain cart or the like. Although the sensors 110 are shown connected with the bus 1606. The connections include, but are not limited to, wired or wireless connections, relayed information between implements, vehicles, a remote or cloud-based portion of the system, a remote or cloud-based information storage device or the like.

In another example, the field monitoring and husbandry system 108 includes a measurement indexing module 1608. In an example, the measurement indexing module 1608 communicates with other components of the system 108 using the bus 1606. The measurement indexing module 1608 indexes measurements provided by the system 108, for instance measurements of one or more of the field characteristics (above-ground, below-ground, or both). In yet another example, the measurement indexing module 1608 indexes measurements to associated portions of the field, for instance by indexing measurements to the zones 900 (shown in FIG. 9A) or sub-zones within the zones 900.

Optionally, the field monitoring and husbandry system 108 includes a geolocation sensor 1610, such as a GPS antenna, real time kinematics (RTK) system or the like. For instance, the measurement indexing module 1608 cooperates with the geolocation sensor 1610 to identify the locations of measurements in the agricultural field 106 to permit indexing of those measurements to the associated zones, sub-zones or the like. For instance, the geolocation sensor 1610 identifies the location of the field sensors 110 with respect to the portions of the field 106, such as the location of the sensors 110 within a specific zone of the zones 900 shown in FIG. 9A, location of the sensors 110 within a sub-zone, or the like as the sensors 110 conduct measurements. In an example, the measurement indexing module 1608 uses the geolocation sensor 1610 to associate measured field characteristics with a zone (or sub-zone) corresponding to the location the field characteristics were measured.

FIG. 9A shows the measured field characteristic (in this example nitrogen content) indexed to the zone where the field characteristic is measured. FIG. 9A shows an example mapping of nitrogen content indexed to portions of the field 106. In some examples, the system 108 generates a map of the field 106 including the measured field characteristics indexed to portions of the field 106. For example, FIG. 9A shows X ppm of nitrogen content indexed to zone 6A. In another example, FIG. 9A shows each of X ppm, X−5 ppm, X+5 ppm, X−8 ppm, X−12 ppm, and X−20 ppm indexed to associated sub-zones of zone 1A. In another example, FIG. 11 shows the shows the measured field characteristic (in this example nitrogen content) indexed to the zone (or sub-zone, or portion) of the field 106 where the field characteristic is measured. For instance, FIG. 11 shows X−5 ppm of nitrogen content indexed to zone 6A. In yet another example, the nitrogen content is included in a first set of field characteristics, and the system 108 indexes a second set of field characteristics, for instance potassium content or the like, with one or more associated portions of the agricultural field. FIG. 9B shows an example mapping of potassium content indexed to portions of the field 106. In a further example, the nitrogen content at the beginning of the season (shown in FIG. 9A) is included in a first set of field characteristics, and the nitrogen content at planting (shown in FIG. 10 is included in a second set of field characteristics. As discussed herein, measurements of field characteristics facilitates determination of the effects of agricultural product applications, changes in nutrients or minerals (including depletion or consumption of nutrients or minerals from applications), determinations of rates (e.g., trends, or the like) for changes, and associations of measured field characteristics (and their changes) with other values, such as yield in the same zones or sub-zones.

Referring to FIG. 16A, the field monitoring and husbandry system 108 optionally includes a yield monitor 1612 or is in communication with a yield monitor 1612 (e.g., provided with a combine, grain cart or the like). The yield monitor 1612 measures one or more yield values for crops grown in the agricultural field 106. FIG. 15 shows an example yield value plotting (ranging between one and ten) for each zone (or sub-zone) within the agricultural field 106. In another example, the measurement indexing module 1608 indexes the measured one or more yield values with portions of the agricultural field 106. For example, the measurement indexing module 1608 indexes yield values to the zones 900 (shown in FIG. 9A) or sub-zones within the zones 900. For instance, the measurement indexing module 1608 cooperates with the geolocation sensor 1610 to determine the locations of measured yield values in the agricultural field 106. In an example, the measurement indexing module 1608 uses the geolocation sensor 1610 to associate measured yield values with a zone where the yield values are measured. In other examples including yield monitors on other implements or vehicles, yield values are measured and indexed with their location, for instance using GPS or RTK sensors provided on those vehicles.

In another example, the yield values (measured with the yield monitor 1612) are associated with one or more of the measured field characteristics (examples shown in FIG. 9, 10, 11, or 12). The field monitoring and husbandry system 108 in FIG. 16A and shown in additional detail in FIG. 16B associates the yield values for the zones 900 (including sub-zones) with the nitrogen content of the zones 900 measured with agricultural implements (e.g., the implement 102 shown in FIG. 1, or the like). Accordingly, the system 108 measures one or more field characteristics over the course of one or more seasons, and associates the yield value for crops with the measured field characteristics in the same zones (including sub-zones). Thus, the system 108 monitors the yield values, and associates the yield values with field characteristics from the same zones (including sub-zones) that achieved the yield values.

In some examples, the field monitoring and husbandry system 108 chooses a favored yield value, the user chooses a favor yield value or the like to facilitate automated refinement of husbandry in the future. The favored yield value corresponds with at least one of the yield values measured with the yield monitor 1612. In an example, the favored yield value is the greatest yield value measured in the field. In another example, the favored yield value is not the greatest yield value, for instance a ten in FIG. 15, and is instead an enhanced yield value achievable in the future with moderated agricultural product application.

In one example, the greatest yield value corresponds with portions of the agricultural field 106 that received a large quantity of agricultural product (relative to other portions) and produced the greatest yield value. Accordingly, the greatest yield value is not necessarily a favored yield value because the price (e.g., labor, agricultural product, or the like) to produce the greatest yield is considered cost prohibitive. Because the system 108 monitors field characteristics and the amount of agricultural product applied throughout a season (or seasons), the system 108 is capable of monitoring and facilitating identification of relatively greater amounts of agricultural product (or labor) that achieved the greatest yield value. Thus, in some examples, the system 108 selects a favored yield value as a target that is less than the greatest yield value but is still a relatively greater yield target for the field, for instance a yield value of nine (instead of ten). In another example, the favored yield value is equal to the greatest yield value, such as the value 10 shown in FIG. 15. For example, the greatest yield value is achieved without application of significant agricultural product or the user determines the application of agricultural product warrants the selection of the greatest yield. In this example, the system 108 selects the greatest yield value as the favored yield value, such as one of the 10 values shown in FIG. 15. In another example, the system collects an array of the greatest yield values from the field and compares those yield values and the agricultural products applied in those same zones. The system 108 selects the greatest yield of the array having the least costly set of associated agricultural products applied as the favored yield value.

In one example, the field characteristic comparator 1620 compares measured yield values, for instance to determine the favored yield value. In yet another example, the system 108 and controller 1600 with the threshold setting module 1624 selects a favored yield value, for instance a yield value of ten in zones 4G, 4H (shown in FIG. 15). The nitrogen content of zones 4G, 4H was measured at harvest to be greater than or equal to X+5 ppm (shown in FIG. 14). In contrast, zones 5G, 5H have a yield value of nine (shown in FIG. 15), and nitrogen content of X ppm at harvest (shown in FIG. 14). In some examples, the system 108 compares (e.g., using the agricultural product module 1622, or the like) the amount of agricultural product applied to zones 4G, 4H with the amount of agricultural product applied to zones 5H, 5H over the course of the growing season. For instance, the system 108 determines that slightly more agricultural product was applied to zones 4G, 4H than applied to zones 5G, 5H. In this example, the slightly more agricultural product applied to zones 4G, 4H is not an excessive amount of agricultural product to achieve a corresponding increase in yield between zones 4G, 4H and zones 5G, 5H. Thus, the system 108 selects the yield value of 10 as the favored yield value.

As further shown in FIG. 16B, the field husbandry controller 1600 includes include a threshold setting module 1624. In one example, the threshold setting module 1624 updates one or more field thresholds. For instance, the threshold setting module 1624 updates a field threshold (e.g., X ppm, shown in FIG. 12) with field characteristics associated with a favored yield value as determined herein. In another example, the field monitoring and husbandry system 108 uses the favored yield value as an input for changes to the field characteristics.

The threshold setting module 1624 uses the field characteristics (or field thresholds) from portions of the agricultural field 106 having the favored yield value to update the field thresholds for other portions of the agricultural field 106. For instance, the system 108 imputes the field characteristics (or field thresholds) associated with the favored yield value across the all or part of the agricultural field. In another example, the threshold setting module 1624 updates the field thresholds for zones 1A-8H to be greater than or equal to X+5 ppm (in contrast to the field thresholds shown in FIG. 12). Thus, other portions of the field that had different yields than the favored yield value have their prescriptions (field thresholds, agricultural product applications or the like) updated to guide the field characteristics for those other portions toward the field characteristics (or field thresholds) associated with the favored yield value. As a result, the system 108 uses the favored yield value and its associated field characteristics to control field characteristics and enhance crop growth in other portions of the field. In another example, the system 108 uses the updated field thresholds (e.g., X+5 ppm or greater) in a following growing season. Consequently, the system 108 uses one or more of the monitored field characteristics, the agricultural product applied to the field 106, or the yield values for crops grown in the field 106 to inform future husbandry of the agricultural field 106. Thus, the system 108 enhance growth, yield, or the like of the crops in the field.

In an embodiment, the system 108 selects the favored yield based on information from a plurality of growing seasons. For instance, the system 108 selects the favored yield based on yield values from two or more growing seasons. In an example, a first growing season is in a first calendar year (e.g., the year 2000, or the like), and the first growing season had a first favored yield value. In another example, a second growing season is in a second calendar year (e.g., the year 2022, or the like), and the second growing season had a second favored yield that is greater than the first favored yield. The system 108 optionally selects the second favored yield to inform future husbandry of the agricultural field, for instance in a third calendar year (e.g., the year 2023, or the like). In another example, the system selects the first favored yield or the second favored yield based on costs to achieve the first favored yield or the second favored yield. In yet another example, the system selects the first favored yield or the second favored yield based on climate in the first calendar year or the second calendar year. For example, the first calendar year was considered to be dry in comparison to the second calendar season that is considered to be wet. In this example, the third calendar year is forecasted to be wet. Accordingly, the system 108 selects the second favored yield because the climate in the second calendar year is similar to the forecast for the (forthcoming) third calendar year. Thus, the system 108 uses climate to inform future husbandry of the field 106. For example, the system imputes field characteristics (or field thresholds) from the second calendar year to the agricultural field 106 in the third calendar year based on selection of the second favored yield value.

As previously discussed herein, other characteristics and associated thresholds for other nutrients, minerals or the like are in some examples also controlled with the system 108. With additional characteristics and associated thresholds the system 108, such as the threshold setting module 1624, similarly updates prescriptions (field thresholds, agricultural product applications associated with the nutrient or mineral or the like) based on the favored yield target and associated values for the other nutrients, minerals or the like. Accordingly, an overall prescription for multiple agricultural products is updated based on the analysis of the favored yield target and its associated field characteristics, product applications or the like.

FIG. 16A shows weight module 1614 in communication with one or more of the field sensors 110. In an example, the weight module 1614 assigns a weight to measurements of the field sensors 110. For instance, the weight module 1614 changes gain applied to measured field characteristics, or electrical signal(s) representative of the measured field characteristics, in correspondence to the weight assigned to the measurements. In another example, the weight module 1614 assigns a first weight to the below-ground field characteristics measured with the stationary below-ground field sensor 700. In yet another example, the weight module 1614 assigns a second weight to the below-ground field characteristics measured with the (on-board) below-ground field sensors 114. In still yet another example, the weight module 1614 assigns a third weight to the above-ground field characteristics measured with the (on-board) above-ground field sensors 112. The weights for the various sensors and associated modules 1614 optionally vary based on one or more factors including, but not limited to, proximity of an implement (having sensors) to a below ground field sensor, a below-ground sensor having a greater weight than an above-ground sensor (or the converse for the above-ground sensor), or the like.

In one example, the system 108 changes one or more of the first weight, second weight, or third weight based on the location of the below-ground field sensors 114 or above-ground field sensors 112 relative to stationary below-ground field sensors 700. For example, the first weight, second weight, or third weight are varied (by the weight module 1614) in proportion to distance of the (on-board) below or above-ground field sensors 114, 112 to the stationary below-ground field sensor 700. In some examples the stationary below-ground field sensor 700 provides enhanced precision, accuracy or both relative to the sensors 112, 114, and accordingly while the vehicle having those sensors 112, 114 is in proximity to the stationary sensor 700 the weights are varied in a manner that favors the stationary sensor 700. Accordingly, the system 108 assigns weight to measurements from the above-ground field sensors 112 or the below-ground field sensors 114 mounted with the implement 102 (or the vehicle 100) based on proximity of the (on-board) sensors 112, 114 to the stationary below-ground field sensor 700. Optionally, the system 108 uses the geolocation sensor 1610 (or a plurality of geolocation sensors, including the geolocation sensor 1610) to determine proximity of the (on-board) sensors 112, 114 in relation to the stationary below-ground field sensor 700.

As noted herein, the stationary below-ground field sensor 700 optionally has enhanced accuracy or precision (or both) in comparison to one or more of the above-ground field sensors 112 or the below-ground field sensors 114 mounted with the implement 102. Accordingly, the system 108 favors (e.g., weights, prioritizes, promotes, or the like) measurements obtained from the stationary below-ground field sensor 700 while the implement 102 (having the above-ground field sensors 112 or the below-ground field sensors 114) is proximate to the stationary below-ground field sensor 700. In contrast, as the vehicle or implement and its sensors are relatively remote from the stationary sensor 700 its measurements are less accurate relative to measurements conducted with the sensors 112, 114 at their present location, and accordingly the sensors 112, 114 are favored and thereby weighted higher. Thus, the accuracy or precision (or both) of measured field characteristics is enhanced by weighting of the measurements provided by the field sensors 110 based on proximity to the stationary below-ground field sensors 700 (where present).

For example, the weight module 1614 (shown in FIG. 16A) changes the first weight for below-ground field characteristics measured by the stationary below-ground field sensor 700 to “1” with the (on-board) sensors 112, 114 proximate to the stationary below-ground field sensor 700, for instance within 10 feet, 20 feet, 50 feet, in the same zone or sub-zone (see FIG. 9A) or the like. Accordingly, one or more of the second weight or the third weight for field characteristics measured with the on-board sensors 112, 114 is changed to “0” (or a lesser weight such 0.25, 0.4 or the like) with the on-board sensors 112, 114 proximate to the stationary below-ground field sensor 700. Optionally, the weights are dynamic and vary in proportion to the distance between the stationary sensor 700 and the vehicle or implement having the associated sensors 112, 114.

For instance, the weight module 1614 changes the first weight (for below-ground field characteristics measured by the stationary below-ground field sensor 700) to “0.25” with the (on-board) sensors 112, 114 near the stationary below-ground field sensor 700. Accordingly, one or more of the second weight or the third weight (for field characteristics measured with the on-board sensors 112, 114) is changed to “0.75” with the (on-board) sensors 112, 114 near the stationary below-ground field sensor 700. In an example, the (on-board) sensors 112, 114 are near the stationary below-ground field sensor 700 in correspondence with the (on-board) sensors 112, 114 near the zone where the stationary below-ground field sensor 700 (e.g., zone 7B, shown in FIG. 9A). FIG. 9A shows the (on-board) sensors 112, 114 in a zone near (e.g., in zone 6G) the stationary below-ground field sensor 700 in zone 7B. In yet another example, and referring to FIG. 9A, the first weight is changed to “0.75” in correspondence with the (on-board) sensors 112, 114 in zone 7A and the stationary below-ground field sensor 700 in zone 7B. Consequently, one or more of the second weight or the third weight is changed to “0.25” in correspondence with the (on-board) sensors 112, 114 in zone 7A and the stationary below-ground field sensor 700 in zone 7B.

In a further example, the weight module 1614 changes the first weight (for below-ground field characteristics measured by the stationary below-ground field sensor 700) to “0” with the (on-board) sensors 112, 114 remote from the stationary below-ground field sensor 700. Accordingly, one or more of the second weight or the third weight (for field characteristics measured with the on-board sensors 112, 114) is changed to “1” with the (on-board) sensors 112, 114 remote from the stationary below-ground field sensor 700. For instance, the (on-board) sensors 112, 114 are remote from the stationary below-ground field sensor 700 in correspondence with the (on-board) sensors 112, 114 in a zone (e.g., in zone 1H) remote from the zone where the stationary below-ground field sensor 700 is stationed (e.g., zone 7B, shown in FIG. 9A).

In another example, the measurements provided by the below-ground field sensors 114 are assigned a higher weight than the measurements provided by the above-ground field sensor 112. In some examples, the below-ground field sensors 114 are more accurate or precise than the above-ground field sensors 112. Accordingly, the weight module 1614 assigns a higher weight to the (more accurate or precise) measurements provided by the below-ground field sensors 114. In yet another example, a correction is applied to measurements provided by the above-ground field sensors 112. Thus, the weight module 1614 increases the weight assigned to the (corrected) measurements provided by the above-ground field sensors 112 (in comparison to uncorrected measurements provided by the above-ground field sensors 112). For instance, the weight module 1614 optionally assigns equal weight (e.g., 0.5, or the like) to the corrected measurements provided by the above-ground field sensors 112 and the measurements provided by the below-ground field sensors 114.

FIG. 16A shows the field husbandry controller includes one or more modules 1616. The one or more modules 1616 of the field husbandry controller 1600 cooperate and permit operation of the field monitoring and husbandry system 108. In an example, the field characteristic comparator 1620 facilitates comparison of field characteristics. For example, the field characteristic comparator 1620 compares measured field characteristics over the course of a season (or seasons). In another example, the agricultural product module 1622 generates an application rate for agricultural product. In yet another example, the field sensor calibration module 1626 facilitates calibration of the field sensors 110.

In another example, the one or more modules 1616 are included in different portions of the system 108. For instance, the modules 1616 (or portions of the modules 1616) are included on-board the vehicle 100 or the implement 102 (shown in FIG. 1). In another example, the modules 1616 are provided as a software add-on to an existing controller for the vehicle 100 or the implement 102. In yet another example, the 1616 are included as hardwired circuitry that communicates with the existing controller for the vehicle 100 or the implement 102. In another example, the field husbandry controller 1600 cooperates with a cloud computing device to operate the system 108. For instance, a first module (e.g., the field threshold module 1618, or the like) is included in the cloud computing device located remote from the agricultural field 106. The field husbandry controller 1600 includes a second module (e.g., the field characteristic comparator 1620, or the like) is on-board the vehicle 100 or the implement 102 and located near the agricultural field 106.

FIG. 16B shows a detailed schematic view of example architecture of the field husbandry controller 1600 at the box 16B of FIG. 16A. The field husbandry controller 1600 includes the one or more modules 1616. In an example, the one or more modules includes a field threshold module 1618. The field threshold module 1618 has one or more field thresholds (e.g., nitrogen content of X ppm, shown in FIG. 12) indexed with the one or more associated portions (e.g., zones 900, shown in FIG. 12) of the agricultural field 106 (shown in FIG. 12). As discussed herein, the field thresholds are, in various examples, compared against measured field characteristics such as nutrients and minerals relative to determine specified application rates for agricultural products to address deviations in nutrients and minerals from the field thresholds.

In another example, the one or more modules 1616 includes a field characteristic comparator 1620. In one example, the field characteristic comparator 1620 facilitates comparison of field characteristics. For instance, the field characteristic comparator 1620 compares field characteristics with one or more field thresholds. In an example, the field characteristic comparator 1620 compares the field characteristics before application (e.g., nitrogen content, shown in FIG. 11) of an agricultural product with one or more field thresholds (e.g., X ppm, shown in FIG. 12). In an example, the field characteristic comparator 1620 determines a characteristic deviation based on the comparison of the field characteristics with the field thresholds. As discussed herein the system 108 addresses the characteristic deviation through application of an agricultural product in a quantity proportional to the deviation.

In yet another example, the nitrogen content at the beginning of the season (shown in FIG. 9A) is included in a first set of field characteristics, and the nitrogen content at planting (shown in FIG. 10) is included in a second set of field characteristics. The field characteristic comparator 1620 optionally compares the first set of field characteristics with the second set of field characteristics. Accordingly, the field characteristic comparator 1620 optionally compares field characteristics before planting of crops (e.g., nitrogen content, shown in FIG. 9A) with field characteristics during planting of crops (e.g., nitrogen content, shown in FIG. 10).

The field husbandry controller 1600 includes an agricultural product module 1622 in another example. The agricultural product module 1622 generates one or more application rates for an agricultural product. For instance, the application rate (e.g., nitrogen content in pounds per acre as one example shown in FIG. 13) is generated based on a comparison of field characteristics for a portion of a field (e.g., X−5 ppm of nitrogen content for zone 8A, shown in FIG. 11) with a field threshold associated with the portion of the field (e.g., X ppm of nitrogen content for zone 8A, shown in FIG. 12). Accordingly, the agricultural product module 1622 generates the one or more application rates based on the determined deviation between the measurement and threshold. The agricultural product applied at the application rate(s) decreases the deviation (including, minimizing, guiding to a deviation of zero, decreasing a difference or the like) between field characteristics and the field thresholds. For instance, the agricultural product module 1622 (shown in FIG. 16B) cooperates with the applicator interface 1604 (shown in FIG. 16A) to operate the agricultural product applicator 1602 (shown in FIG. 16A) and apply the agricultural product (or agricultural products) at the application rate.

In another example, the agricultural product module 1622 optionally uses real time or near real time measured field characteristics to update or refine application rates to achieve the field thresholds. For example, the system 108 includes the field sensors 110 directed in front of the agricultural vehicle 100. The field sensors 110 measure field characteristics as the vehicle 100 (such as a sprayer) navigates the field 106 (shown in FIG. 1). The system 108 optionally uses the field characteristics measured by field sensors 110 mounted with the sprayer 102E to update or refine the application rate (or rates) of agricultural product. In some examples, the field characteristics vary from earlier measurements (e.g., the measured field characteristics shown in FIG. 9A) based on time between the measurements and application of agricultural product. For instance, weather changes (e.g., rain, drought, or the like) potentially cause changes in the field characteristics after measurement of the field characteristics. Accordingly, the deviation between the field thresholds and the earlier measured field characteristics varies from what was originally determined (based on the earlier measurement of field characteristics). Thus, with field characteristics that have decreased additionally in the intervening span of time the application rate that is based on the earlier measurement of field characteristics is insufficient to achieve the desired field threshold. As a result, the system 108 updates the field characteristic measurements (potentially in near real time) with the field sensors, and refines the application rate to achieve the field threshold based on the updated field characteristics.

For example, the cultivator 102C (shown in FIG. 3) has field sensors 110 that measure field characteristics after crops are planted in the field 106. The sprayer 102E (shown in FIG. 5) has field sensors 110 that measure field characteristics as the sprayer 102E navigates the field 106 after the cultivator 102C. The system 108 generates application rates based on one or more of the measurements provided by the field sensors 110 mounted with the cultivator 102C or the sprayer 102E. For instance, the system 108 generates an application rate based on a difference between the field characteristics that were measured with the sprayer 102E and the field characteristics measured with the cultivator 102C. As described herein (e.g., above), the field characteristics vary in some examples based on time between the cultivator operation and sprayer operation, for instance because of weather. Accordingly, in some examples, the application rates determined with the field characteristics previously measured with the cultivator 102C (the earlier operation) are insufficient to achieve the specified field thresholds. In this example, the system 108 updates the application rates based on more contemporary measurements provided by field sensors 110 mounted with the sprayer 102E. For example, the field sensors 110 directed ahead of (e.g., forward to or into the page of FIG. 5) the booms 500 of the sprayer 102E. The field sensors 110 measure the field characteristics before the sprayer 102E applies the agricultural product. The system 108 updates the previous measurements with the sensor 110 measurements or optionally refines the previous measurements. The system 108 then updates the application rate (from the rate originally determined based on field characteristics measured with the cultivator 102C) based on the recent measurements obtained by the field sensors 110 mounted with the sprayer 102E to achieve the field thresholds. As a result, the system 108 achieves the specified field thresholds even with intervening changes to the field characteristics prior to application of an agricultural product (and prior to determining the application rate).

In a further example, the field husbandry controller 1600 shown in FIG. 16B includes a field sensor calibration module 1626. The field sensor calibration module 1626 calibrates one or more of the field sensors 110 with measurement compensation as described herein. In some approaches, the above-ground field sensors 112 have limited accuracy, precision or both relative to a below-ground field sensors (e.g., one or more of on-board below-ground field sensors 114 or the stationary below-ground field sensor 700). For example, the below-ground field sensors 114, 700 include sensor elements below ground and are accordingly (at least initially) able to provide greater precision and accuracy for field characteristic measurements of nutrients, minerals or the like within the soil. In contrast, above-ground field sensors 112 may, in some examples, be able to sense one or more of those nutrients, minerals or the like, however the sensing is potentially less accurate or less precise. In an example, the system 108 includes the sensor calibration module 1626 to calibrate the above-ground field sensors 112 to enhance the accuracy, precision, or both of the above-ground field sensors 112 and facilitate their use without below-ground counterpart sensors 114, 700. The field sensor calibration module 1626 calibrates the above-ground field sensors 112 based on below-ground field characteristics (measured by a below-ground field sensor 114, 700). With the calibration the above-ground field sensors 112 provide precise and accurate measurements that emulate the precision and accuracy of below-ground field sensors (e.g., the below-ground field sensors 114).

In one example, the field sensor calibration module 1626 of the system 108 marries measurements of the below-ground field sensors 114 with corresponding measurements of the above-ground field sensors 112. Both sensors 112, 114 are provided with the moving vehicle or implement and thereby are sensing approximately the same targets (including similar or proximate targets), such as the soil of a zone, sub-zone, or higher resolution portion of the field (e.g., 1 square feet, 3 square feet or the like). As shown in previous examples herein (see FIGS. 1-4) the above-ground sensors 112 are provided along the same or proximate line travel lines of the below-ground sensor 114 counterparts. Sensing of similar targets and conducting sensing of those targets in an ongoing manner permits ongoing comparison of like below-ground and above-ground measurements to determine measurement calibrations.

The field sensor calibration module 1626 adjusts the above-ground field characteristic sensing in a manner commensurate to the (more precise or accurate) below-ground field characteristic sensing. In yet another example, the field characteristic comparator 1620 compares the above-ground measured field characteristics with the below-ground measured field characteristics (e.g., taken at approximately the same locations). The comparisons determine a sensor-based measurement deviation (also referred to a sensor deviation). The field sensor calibration module 1626 generates the measurement compensation value based on the sensor deviation. The field sensor calibration module 1626 applies the measurement compensation value to ongoing measurements of the above-ground field characteristics to provide enhanced sensing with the above-ground sensors 112 that emulates the accuracy, precision or both of below-ground sensors (e.g., one or more of the below-ground field sensors 114 or the stationary below-ground field sensor 700).

Thus, the field sensor calibration module 1626 calibrates the above-ground sensors 112 with preceding measurements of field characteristics with the below-ground field sensors 114 and the above-ground sensors 112. Accordingly, establishes a correspondence between the measured above-ground field characteristics and the measured below-ground field characteristics. In another example, the field sensor calibration module 1626 brings the above-ground field characteristics into accordance with the below-ground field characteristics. Because, in some examples, the above and below ground sensors 112, 114 move together (e.g., are mounted with the vehicle or implement 102, shown in FIG. 1) the measurement compensation is readily determined based on sensor deviations as the sensors 112, 114 move through the agricultural field 106 (shown in FIG. 1). In another example, the below-ground field sensors 114 is mounted with a first implement (e.g., a cultivator, or the like), and the above-ground field sensors 112 is mounted with a second implement (e.g., a sprayer, or the like). The measurements of both sensors 114, 112 are indexed to their respective locations in the field (e.g., zones, sub-zones, higher resolution portions or the like). The comparisons discussed above, determinations of deviations and the like are conducted between measurements from the same indexed locations (including similar or proximate locations). The system 108 having the field sensor calibration module 1626 accordingly uses the indexed below-ground measurements and the indexed above-ground measurements to determine a measurement compensation based on deviations therebetween as noted above.

As a result of calibrating the above-ground field sensors 112, the system 108 enhances the accuracy (precision, or both) of the above-ground field sensing and monitored field characteristics using the above-ground field sensors 112. For example, a vehicle (e.g., a sprayer, or the like) that includes the above-ground field sensors 112 but does not include the below-ground field sensors 114 uses the calibration (e.g., one or more measurement compensation values) to enhance the accuracy (or precision) of above-ground field characteristics measured with the above-ground field sensors 112. Optionally, the measurement compensation values are similarly indexed to zones, sub-zones or the like in the field, and as the vehicle enters those locations the measurement compensation value for that portion is used with above-ground field sensor 112. Accordingly, measurement compensation values determined in various portions of the field are then used in those portions for above-ground sensing to further enhance precision and accuracy. In another example with the implement 102 as a sprayer, the sprayer does not engage with field soil, and accordingly the sprayer does not include the below-ground field sensors 114. Thus, the system 108 accurately and precisely measures field characteristics in a manner approaching below-ground sensors without disturbing the soil or requiring instruments that penetrate the soil, for example with crops nearing harvest growing in the soil. Using the calibration provided by the field sensor calibration module 1626, implements and vehicles such as harvesters (combines), sprayers or the like, that do not include below-ground implements (e.g., a ripper, cultivator, or the like) include above-ground sensors 112 that readily provide accurate and precise field characteristic measurements enhanced with the measurement compensation value (or values) previously determined by the field sensor calibration module 1626. Accordingly, accuracy, precision, or both of above-ground field characteristics are enhanced with the field monitoring and husbandry system 108.

In another example, the one or more modules 1616 includes a map generation module 1628. The map generation module 1628 generates maps of the field 106. For example, the map generation module 1628 generates a map of the measured field characteristics indexed to portions of the field (e.g., one or more the field characteristics shown in FIGS. 9A, 9B, 10, 11, or 14, or the like). In another example, the map generation module 1628 generates a map of the measured field characteristics indexed to portions of the field (e.g., the yield values shown in FIGS. 15, or the like). In yet another example, the map generation module 1628 generates a map of the field thresholds indexed to portions of the field (e.g., the field thresholds shown in FIGS. 12, or the like). In a further example, the map generation module 1628 generates a map of the application rates for agricultural product(s) indexed to portions of the field (e.g., the application rates shown in FIGS. 13, or the like). Accordingly, the system 108 provides a user with information regarding one or more of the field characteristics, husbandry to the field 106, or crops grown in the field 106.

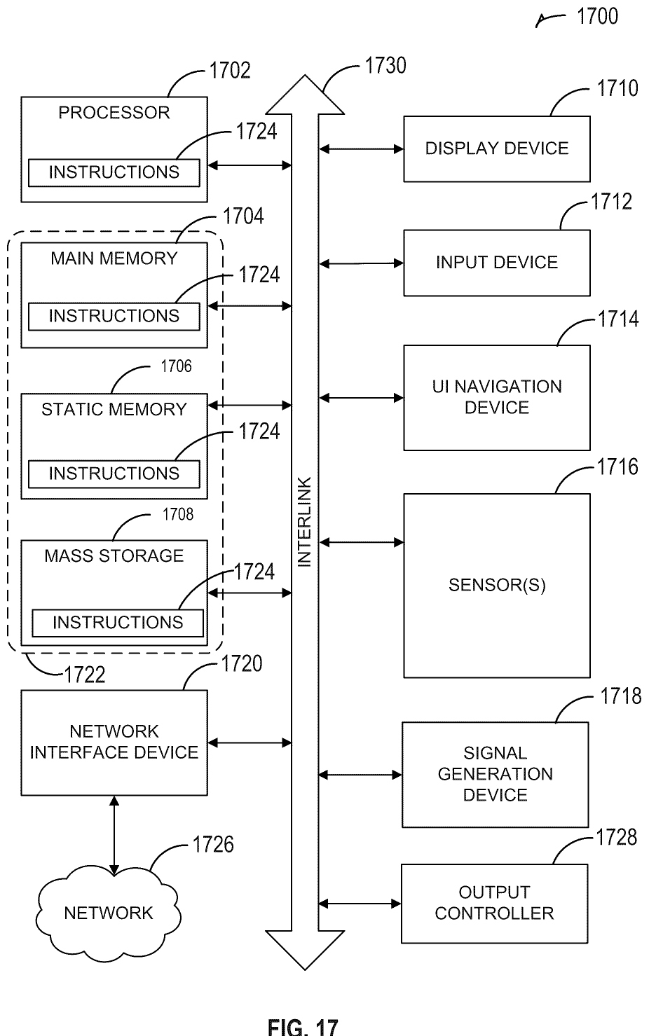

FIG. 17 illustrates a block diagram of an example machine 1700 upon which any one or more of the techniques (e.g., methodologies) discussed herein may perform. Examples, as described herein, may include, or may operate by, logic or a number of components, or mechanisms in the machine 1700. Circuitry (e.g., processing circuitry) is a collection of circuits implemented in tangible entities of the machine 1700 that include hardware (e.g., simple circuits, gates, logic, etc.). Circuitry membership may be flexible over time. Circuitries include members that may, alone or in combination, perform specified operations when operating. In an example, hardware of the circuitry may be immutably designed to carry out a specific operation (e.g., hardwired). In an example, the hardware of the circuitry may include variably connected physical components (e.g., execution units, transistors, simple circuits, etc.) including a machine readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuitry in hardware via the variable connections to carry out portions of the specific operation when in operation. Accordingly, in an example, the machine-readable medium elements are part of the circuitry or are communicatively coupled to the other components of the circuitry when the device is operating. In an example, any of the physical components may be used in more than one member of more than one circuitry. For example, under operation, execution units may be used in a first circuit of a first circuitry at one point in time and reused by a second circuit in the first circuitry, or by a third circuit in a second circuitry at a different time. Additional examples of these components with respect to the machine 1700 follow.

In alternative embodiments, the machine 1700 may operate as a standalone device or may be connected (e.g., networked) to other machines. In a networked deployment, the machine 1700 may operate in the capacity of a server machine, a client machine, or both in server-client network environments. In an example, the machine 1700 may act as a peer machine in peer-to-peer (P2P) (or other distributed) network environment. The machine 1700 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

The machine (e.g., computer system) 1700 may include a hardware processor 1702 (e.g., a central processing unit (CPU), a graphics processing unit (GPU), a hardware processor core, or any combination thereof), a main memory 1704, a static memory (e.g., memory or storage for firmware, microcode, a basic-input-output (BIOS), unified extensible firmware interface (UEFI), etc.) 1706, and mass storage 1708 (e.g., hard drive, tape drive, flash storage, or other block devices) some or all of which may communicate with each other via an interlink (e.g., bus) 1730. The machine 1700 may further include a display unit 1710, an alphanumeric input device 1712 (e.g., a keyboard), and a user interface (UI) navigation device 1714 (e.g., a mouse). In an example, the display unit 1710, input device 1712 and UI navigation device 1714 may be a touch screen display. The machine 1700 may additionally include a storage device (e.g., drive unit) 1708, a signal generation device 1718 (e.g., a speaker), a network interface device 1720, and one or more sensors 1716, such as a global positioning system (GPS) sensor, compass, accelerometer, or other sensor. The machine 1700 may include an output controller 1728, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

Registers of the processor 1702, the main memory 1704, the static memory 1706, or the mass storage 1708 may be, or include, a machine-readable medium 1722 on which is stored one or more sets of data structures or instructions 1724 (e.g., software) embodying or utilized by any one or more of the techniques or functions described herein. The instructions 1724 may also reside, completely or at least partially, within any of registers of the processor 1702, the main memory 1704, the static memory 1706, or the mass storage 1708 during execution thereof by the machine 1700. In an example, one or any combination of the hardware processor 1702, the main memory 1704, the static memory 1706, or the mass storage 1708 may constitute the machine-readable media 1722. While the machine readable medium 1722 is illustrated as a single medium, the term “machine readable medium” may include a single medium or multiple media (e.g., a centralized or distributed database, and/or associated caches and servers) configured to store the one or more instructions 1724.

The term “machine readable medium” may include any medium that is capable of storing, encoding, or carrying instructions for execution by the machine 1700 and that cause the machine 1700 to perform any one or more of the techniques of the present disclosure, or that is capable of storing, encoding or carrying data structures used by or associated with such instructions. Non-limiting machine-readable medium examples may include solid-state memories, optical media, magnetic media, and signals (e.g., radio frequency signals, other photon-based signals, sound signals, etc.). In an example, a non-transitory machine-readable medium comprises a machine-readable medium with a plurality of particles having invariant (e.g., rest) mass, and thus are compositions of matter. Accordingly, non-transitory machine-readable media are machine readable media that do not include transitory propagating signals. Specific examples of non-transitory machine-readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 1724 may be further transmitted or received over a communications network 1726 using a transmission medium via the network interface device 1720 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.17.4 family of standards, peer-to-peer (P2P) networks, among others. In an example, the network interface device 1720 may include one or more physical jacks (e.g., Ethernet, coaxial, or phone jacks) or one or more antennas to connect to the communications network 1726. In an example, the network interface device 1720 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 1700, and includes digital or analog communications signals or other intangible medium to facilitate communication of such software. A transmission medium is a machine-readable medium.

### Various Notes & Examples

Example 1 is a field monitoring and husbandry system configured for mounting with one or more of an agricultural vehicle or an agricultural implement, the system comprising: at least one field sensor configured for mounting to the agricultural vehicle or the agricultural implement, the at least one field sensor configured to measure one or more field characteristics within an agricultural field; a measurement indexing module in communication with the at least one field sensor, the measurement indexing module configured to index measurements of the at least one field sensor with one or more associated portions of the agricultural field; and a field husbandry controller in communication with the at least one field sensor, the field husbandry controller includes: a map generation module configured to generate a map of at least the measurements of the at least one field sensor indexed with the one or more associated portions of the field.

In Example 2, the subject matter of Example 1 optionally includes wherein the field husbandry controller includes: a field threshold module having one or more field thresholds indexed with the one or more associated portions of the agricultural field; a field characteristic comparator configured to compare the one or more field characteristics measured with the at least one field sensor and an associated field threshold of the one or more field thresholds, each of the compared one or more field characteristics and the associated field threshold indexed to a corresponding portion of the one or more associated portions; and an agricultural product module configured to generate an application rate of one or more agricultural products based on the comparison for the corresponding portion.

In Example 3, the subject matter of Example 2 optionally includes wherein the one or more field thresholds includes a plurality of field thresholds, and the associated field threshold includes a first field threshold of the plurality of field thresholds.

In Example 4, the subject matter of any one or more of Examples 1-3 optionally include wherein the one or more associated portions of the agricultural field includes a plurality of associated portion of the agricultural field; and the one or more field characteristics includes at least one or more field characteristics for each associated portion of the plurality of associated portions.

In Example 5, the subject matter of Example 4 optionally includes wherein the one or more field characteristics includes two or more field characteristics for each associated portion of the plurality of associated portions.

In Example 6, the subject matter of any one or more of Examples 2-5 optionally include a yield monitor in communication with the field husbandry controller, the yield monitor configured to measure one or more yield values for the one or more associated portions of the agricultural field.

In Example 7, the subject matter of Example 6 optionally includes wherein the measurement indexing module is in communication with the yield monitor, and the measurement indexing module is configured to index the measured one or more yield values from the one or more associated portions with the one or more associated portions of the agricultural field.

In Example 8, the subject matter of Example 7 optionally includes wherein: the one or more yield values includes a first yield value indexed with a first associated portion of the one or more associated portions; the one or more yield values includes a second yield value indexed with a second associated portion of the one or more associated portions; the field husbandry controller is configured to associate the first yield value with measured field characteristics of the first associated portion; and the field husbandry controller is configured to associate the second yield value with measured field characteristics of the second associated portion.

In Example 9, the subject matter of Example 8 optionally includes wherein the field characteristic comparator is configured to compare the first yield value with the second yield value; and the field husbandry controller is configured to select a favored yield value corresponding to one of the first yield value or the second yield value and associated measured field characteristics based on the comparison.

In Example 10, the subject matter of Example 9 optionally includes wherein the first yield value is greater than the second yield value, and the field husbandry controller is configured to select the first yield value as the favored yield value.

In Example 11, the subject matter of any one or more of Examples 9-10 optionally include wherein the field husbandry controller includes a threshold setting module configured to update at least one of the one or more field thresholds indexed to the one or more associated portions of the field based on the measured field characteristics for the associated portions of the field associated with the favored yield value.

In Example 12, the subject matter of any one or more of Examples 7-11 optionally include wherein: the one or more yield values includes a first yield value indexed with a first associated portion of the one or more associated portions; a first field threshold of the one or more field thresholds is indexed with the first associated portion the one or more yield values includes a second yield value indexed with a second associated portion of the one or more associated portions; a second field threshold of the one or more field thresholds is indexed with the second associated portion the field husbandry controller is configured to associate the first yield value with the first field threshold; and the field husbandry controller is configured to associate the second yield value with the second field threshold.

In Example 13, the subject matter of Example 12 optionally includes wherein the field husbandry controller is configured to compare the first yield value with the second yield value; and the field husbandry controller is configured to select a favored yield value corresponding to one of the first yield value or the second yield value and associated field thresholds based on the comparison.

In Example 14, the subject matter of Example 13 optionally includes wherein the field husbandry controller includes a threshold setting module configured to update at least one of the one or more field thresholds indexed to the one or more associated portions of the field based on field thresholds associated with the favored yield value.

In Example 15, the subject matter of any one or more of Examples 2-14 optionally include an applicator interface in communication with the agricultural product module, wherein the applicator interface is configured to couple with an agricultural product applicator.

In Example 16, the subject matter of Example 15 optionally includes the agricultural product applicator in communication with the applicator interface.

In Example 17, the subject matter of any one or more of Examples 1-16 optionally include wherein the measured field characteristics indexed with the one or more associated portions of the agricultural field are included in a first set of field characteristics, and the measurement indexing module is configured to index a second set of field characteristics with the one or more associated portions of the agricultural field.

In Example 18, the subject matter of Example 17 optionally includes wherein the field husbandry controller is configured to compare the first set of field characteristics with the second set of field characteristics to determine a field characteristic deviation for the one or more associated portions of the agricultural field.

In Example 19, the subject matter of any one or more of Examples 17-18 optionally include wherein the field husbandry controller is configured to determine a rate of change between the first set of field characteristics and the second set of field characteristics for the one or more associated portions of the agricultural field.

In Example 20, the subject matter of any one or more of Examples 17-19 optionally include wherein: the at least one field sensor includes a first field sensor and a second field sensor; the agricultural implement includes a first agricultural implement and a second agricultural implement; the first field sensor is configured for coupling with the first agricultural implement; the second field sensor is configured for coupling with the second agricultural implement; the first field sensor is configured to measure the first set of field characteristics; and the second field sensor is configured to measure the second set of field characteristics.

In Example 21, the subject matter of any one or more of Examples 1-20 optionally include wherein the agricultural implement includes one or more of a tiller, a plow, a planter, a cultivator, a harvester, a swather, a combine, a sprayer, or a trailer.

Example 22 is a field monitoring and husbandry system configured for mounting with one or more of an agricultural vehicle or an agricultural implement, the system comprising: at least one field sensor configured for mounting to the agricultural vehicle or the agricultural implement, the at least one field sensor configured to measure one or more field characteristics within an agricultural field; a yield monitor configured to measure one or more yield values for the one or more associated portions agricultural field; and a field husbandry controller in communication with the at least one field sensor, the field husbandry controller includes: a field threshold module having one or more field thresholds indexed with the one or more associated portions of the agricultural field; a threshold setting module configured to update at least one of the one or more field thresholds indexed to the one or more associated portions of the field based on the one or more yield values; and an agricultural product module configured to generate an application rate of one or more agricultural products based on the updated at least one of the one or more field thresholds compared with the one or more field characteristics.

In Example 23, the subject matter of Example 22 optionally includes an applicator interface in communication with the agricultural product module, wherein the application interface is configured to couple with an agricultural product applicator.

In Example 24, the subject matter of Example 23 optionally includes the agricultural product applicator in communication with the applicator interface.

In Example 25, the subject matter of any one or more of Examples 22-24 optionally include wherein the field husbandry controller includes a field characteristic comparator configured to compare the one or more field characteristics measured with the at least one field sensor and an associated field threshold of the one or more field thresholds, each of the compared one or more field characteristics and the associated field threshold indexed to a corresponding portion of the one or more associated portions.

In Example 26, the subject matter of any one or more of Examples 22-25 optionally include a measurement indexing module in communication with the at least one field sensor, the measurement indexing module configured to index measurements of the at least one field sensor with one or more associated portions of the agricultural field.

In Example 27, the subject matter of Example 26 optionally includes wherein the measurement indexing module is in communication with the yield monitor, and the measurement indexing module is configured to index the measured one or more yield values from the one or more associated portions with the one or more associated portions of the agricultural field.

In Example 28, the subject matter of Example 27 optionally includes wherein: the one or more yield values includes a first yield value indexed with a first associated portion of the one or more associated portions; the one or more yield values includes a second yield value indexed with a second associated portion of the one or more associated portions; the field husbandry controller is configured to associate the first yield value with measured field characteristics of the first associated portion; and the field husbandry controller is configured to associate the second yield value with measured field characteristics of the second associated portion.

In Example 29, the subject matter of Example 28 optionally includes wherein the field husbandry controller is configured to compare the first yield value with the second yield value; and the field husbandry controller is configured to select a favored yield value corresponding to one of the first yield value or the second yield value and associated measured field characteristics based on the comparison.

In Example 30, the subject matter of Example 29 optionally includes wherein the first yield value is greater than the second yield value, and the field husbandry controller is configured to select the first yield value as the favored yield value.

In Example 31, the subject matter of any one or more of Examples 29-30 optionally include wherein the field husbandry controller includes a threshold setting module configured to update at least one of the one or more field thresholds indexed to the one or more associated portions of the field based on the measured field characteristics for the associated portions of the field associated with the favored yield value.

In Example 32, the subject matter of any one or more of Examples 27-31 optionally include wherein: the one or more yield values includes a first yield value indexed with a first associated portion of the one or more associated portions; a first field threshold of the one or more field thresholds is indexed with the first associated portion the one or more yield values includes a second yield value indexed with a second associated portion of the one or more associated portions; a second field threshold of the one or more field thresholds is indexed with the second associated portion the field husbandry controller is configured to associate the first yield value with the first field threshold; and the field husbandry controller is configured to associate the second yield value with the second field threshold.

In Example 33, the subject matter of Example 32 optionally includes wherein the field husbandry controller is configured to compare the first yield value with the second yield value; and the field husbandry controller is configured to select a favored yield value corresponding to one of the first yield value or the second yield value and associated field thresholds based on the comparison.

In Example 34, the subject matter of Example 33 optionally includes wherein the threshold setting module is configured to update the at least one of the one or more field thresholds indexed to the one or more associated portions of the field based on field thresholds associated with the favored yield value.

In Example 35, the subject matter of any one or more of Examples 33-34 optionally include wherein the threshold setting module is configured to update the at least one of the one or more field thresholds indexed to the one or more associated portions of the field based on field characteristics associated with the favored yield value.

In Example 36, the subject matter of any one or more of Examples 27-35 optionally include wherein: the one or more yield values includes: a first yield value indexed to a first zone; a second yield value indexed to a second zone; and a third yield value indexed to a third zone; the yield monitor is configured to compare the first yield value with the second yield value; the yield monitor is configured to compare the first yield value with the third yield value; and the yield monitor is configured to identify a greatest yield value based on the comparisons of the first yield value with each of the second yield value and the third yield value.

Example 37 is a field monitoring and husbandry system configured for mounting with one or more of an agricultural vehicle or an agricultural implement, the system comprising: at least one above-ground field sensor configured for mounting to the agricultural vehicle or the agricultural implement, the at least one above-ground field sensor configured to measure at least a first field characteristic within an agricultural field; at least one below-ground field sensor configured to measure one or more of the first field characteristic or a second field characteristic within the agricultural field; a measurement indexing module in communication with the at least one above-ground field sensor and the at least one below-ground field sensor, the measurement indexing module configured to index measurements of the at least one above-ground field sensor and the at least one below-ground field sensor with one or more associated portions of the agricultural field; and a field husbandry controller in communication with the above-ground field sensor and the below-ground field sensor, the field husbandry controller includes: a field characteristic comparator configured to compare the above-ground characteristics with the below-ground characteristics to determine a sensor deviation between the above-ground characteristics and the below-ground characteristics; and a field sensor calibration module configured to apply a measurement compensation value to the measured one or more above-ground field characteristics based on the sensor deviation.

In Example 38, the subject matter of Example 37 optionally includes wherein: the below-ground field sensor includes a stationary below-ground field sensor configured for stationing in the agricultural field and an on-board below-ground field sensor configured for mounting to the agricultural vehicle; the field husbandry controller is configured to assign a first weight to the below-ground field characteristics measured with the stationary below-ground field sensor; the field husbandry controller is configured to assign a second weight to the below-ground field characteristics measured with the on-board below-ground field sensor; the first weight and the second weight vary in proportion to distance of the above-ground field sensor in relation to the stationary below-ground field sensor; and the measurement compensation value is based on the first weight and the second weight.

In Example 39, the subject matter of any one or more of Examples 37-38 optionally include wherein the at least one above-ground field sensor or the at least one below-ground field sensor configured to determine or more field characteristics includes one or more of a laser sensor, a hyperspectral sensor, a multispectral sensor, an NDVI sensor, a satellite sensor, an ultrasonic sensor, a ground penetrating radar sensor, a gamma ray sensor, an infrared sensor, a temperature sensor, a moisture sensor, or a pH sensor.

In Example 40, the subject matter of any one or more of Examples 37-39 optionally include wherein the field sensor calibration module is configured to apply the measurement compensation to the measured one or more above-ground field characteristics and establish a correspondence between the measured one or more above-ground field characteristics and the measured one or more below-ground field characteristics.

In Example 41, the subject matter of any one or more of Examples 37-40 optionally include wherein the field husbandry controller includes: a map generation module configured to generate a map of at least the measurements of the at least one field sensor indexed with the one or more associated portions of the field.

In Example 42, the subject matter of any one or more of Examples 37-41 optionally include wherein the above-ground field sensor is configured to measure one or more of: the first field characteristic above a surface of soil in the agricultural field, the first field characteristic at the surface, or the first field characteristic below the surface of the soil.

In Example 43, the subject matter of any one or more of Examples 37-42 optionally include wherein the below-ground field sensor is configured to measure one or more of: the second field characteristic at a surface of soil in the agricultural field, or below the surface of the soil.

In Example 44, the subject matter of any one or more of Examples 37-43 optionally include wherein the at least one above-ground field sensor refrains from directly contacting soil in the agricultural field.

In Example 45, the subject matter of any one or more of Examples 37-44 optionally include wherein the at least one below-ground field sensor directly contacts soil in the agricultural field.

Each of these non-limiting examples can stand on its own, or can be combined in various permutations or combinations with one or more of the other examples.

The above description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. A field monitoring and husbandry system configured for mounting with one or more of an agricultural vehicle or an agricultural implement, the system comprising:
at least one field sensor configured for mounting to the agricultural vehicle or the agricultural implement, the at least one field sensor configured to measure one or more field characteristics within an agricultural field;
a measurement indexing module in communication with the at least one field sensor, the measurement indexing module configured to index measurements of the at least one field sensor with one or more associated portions of the agricultural field; and
a field husbandry controller in communication with the at least one field sensor, the field husbandry controller includes:
a map generation module configured to generate a map of at least the measurements of the at least one field sensor indexed with the one or more associated portions of the field.

2. The system of claim 1, wherein the field husbandry controller includes:
a field threshold module having one or more field thresholds indexed with the one or more associated portions of the agricultural field;
a field characteristic comparator configured to compare the one or more field characteristics measured with the at least one field sensor and an associated field threshold of the one or more field thresholds, each of the compared one or more field characteristics and the associated field threshold indexed to a corresponding portion of the one or more associated portions; and
an agricultural product module configured to generate an application rate of one or more agricultural products based on the comparison for the corresponding portion.

3. The system of claim 2, wherein the one or more field thresholds includes a plurality of field thresholds, and the associated field threshold includes a first field threshold of the plurality of field thresholds.

4. The system of claim 1, wherein the one or more associated portions of the agricultural field includes a plurality of associated portion of the agricultural field; and
the one or more field characteristics includes at least one or more field characteristics for each associated portion of the plurality of associated portions.

5. The system of claim 4, wherein the one or more field characteristics includes two or more field characteristics for each associated portion of the plurality of associated portions.

6. The system of claim 2, further comprising a yield monitor in communication with the field husbandry controller, the yield monitor configured to measure one or more yield values for the one or more associated portions of the agricultural field.

7. The system of claim 6, wherein the measurement indexing module is in communication with the yield monitor, and the measurement indexing module is configured to index the measured one or more yield values from the one or more associated portions with the one or more associated portions of the agricultural field.

8. The system of claim 7, wherein:
the one or more yield values includes a first yield value indexed with a first associated portion of the one or more associated portions;
the one or more yield values includes a second yield value indexed with a second associated portion of the one or more associated portions;
the field husbandry controller is configured to associate the first yield value with measured field characteristics of the first associated portion; and
the field husbandry controller is configured to associate the second yield value with measured field characteristics of the second associated portion.

9. The system of claim 8, wherein the field characteristic comparator is configured to compare the first yield value with the second yield value; and
the field husbandry controller is configured to select a favored yield value corresponding to one of the first yield value or the second yield value and associated measured field characteristics based on the comparison.

10. The system of claim 9, wherein the first yield value is greater than the second yield value, and the field husbandry controller is configured to select the first yield value as the favored yield value.

11. The system of claim 9, wherein the field husbandry controller includes a threshold setting module configured to update at least one of the one or more field thresholds indexed to the one or more associated portions of the field based on the measured field characteristics for the associated portions of the field associated with the favored yield value.

12. The system of claim 7, wherein:
the one or more yield values includes a first yield value indexed with a first associated portion of the one or more associated portions;
a first field threshold of the one or more field thresholds is indexed with the first associated portion
the one or more yield values includes a second yield value indexed with a second associated portion of the one or more associated portions;
a second field threshold of the one or more field thresholds is indexed with the second associated portion
the field husbandry controller is configured to associate the first yield value with the first field threshold; and
the field husbandry controller is configured to associate the second yield value with the second field threshold.

13. The system of claim 12, wherein the field husbandry controller is configured to compare the first yield value with the second yield value; and
the field husbandry controller is configured to select a favored yield value corresponding to one of the first yield value or the second yield value and associated field thresholds based on the comparison.

14. The system of claim 13, wherein the field husbandry controller includes a threshold setting module configured to update at least one of the one or more field thresholds indexed to the one or more associated portions of the field based on field thresholds associated with the favored yield value.

15. The system of claim 2, further comprising an applicator interface in communication with the agricultural product module, wherein the applicator interface is configured to couple with an agricultural product applicator.

16. The system of claim 15, further comprising the agricultural product applicator in communication with the applicator interface.

17. The system of claim 1, wherein the measured field characteristics indexed with the one or more associated portions of the agricultural field are included in a first set of field characteristics, and the measurement indexing module is configured to index a second set of field characteristics with the one or more associated portions of the agricultural field.

18. The system of claim 17, wherein the field husbandry controller is configured to compare the first set of field characteristics with the second set of field characteristics to determine a field characteristic deviation for the one or more associated portions of the agricultural field.

19. The system of claim 17, wherein the field husbandry controller is configured to determine a rate of change between the first set of field characteristics and the second set of field characteristics for the one or more associated portions of the agricultural field.

20. The system of claim 17, wherein:
the at least one field sensor includes a first field sensor and a second field sensor;
the agricultural implement includes a first agricultural implement and a second agricultural implement;
the first field sensor is configured for coupling with the first agricultural implement;
the second field sensor is configured for coupling with the second agricultural implement;
the first field sensor is configured to measure the first set of field characteristics; and
the second field sensor is configured to measure the second set of field characteristics.

21. The system of claim 1, wherein the agricultural implement includes one or more of a tiller, a plow, a planter, a cultivator, a harvester, a swather, a combine, a sprayer, or a trailer.
